(19) United States
(12) Patent Application Publication
(10) Pub. No.: US 2019/0224276 A1
(43) Pub. Date: Jul. 25, 2019

(54) ESCALATING DOSAGE SCHEDULES FOR TREATING CELIAC DISEASE

(71) Applicant: ImmusanT, Inc., Cambridge, MA (US)

(72) Inventor: Robert P. Anderson, Shrewsbury, MA (US)

(73) Assignee: ImmusanT, Inc., Cambridge, MA (US)

(21) Appl. No.: 16/311,794

(22) PCT Filed: Jun. 28, 2017
(86) PCT No.: PCT/US2017/039834

§ 371 Date: Dec. 20, 2018

Related U.S. Application Data
(60) Provisional application No. 62/355,860, filed on Jun. 28, 2016.
(60) Provisional application No. 62/458,445, filed on Feb. 13, 2017.

(51) Int. Cl. A61K 38/16; A61P 1/00; A61K 9/00

(52) U.S. Cl. CPC A61K 38/168; A61K 9/0019; A61P 1/00

(57) ABSTRACT

Provided herein are specific dosages and dosage schedules of a composition comprising at least one gluten peptide for use in treating subjects with Celiac disease.

### RELATED APPLICATIONS

This application claims the benefit of priority under 35 U.S.C. § 119(e) of U.S. provisional application No. 62/355,860, filed Jun. 28, 2016, and U.S. provisional application No. 62/458,445, filed Feb. 13, 2017, the entire contents of each of which are incorporated by reference herein.

### BACKGROUND

Celiac disease, also known as coeliac disease or Celiac sprue (Coeliac sprue), affects approximately 1% of people in Europe and North America. In many of those affected, Celiac disease is unrecognised, but this clinical oversight is now being rectified with greater clinical awareness. A gluten free diet is the only currently approved treatment for Celiac disease, and because regular ingestion of as little as 50 mg of gluten (equivalent to 1/100^{th} of a standard slice of bread) can damage the small intestine; chronic inflammation of the small bowel is commonplace in subjects on a gluten free diet. Persistent inflammation of the small intestine has been shown to increase the risk of cancer, osteoporosis and death. As gluten is so widely used, for example, in commercial soups, sauces, ice-creams, etc., maintaining a gluten-free diet is difficult.

Celiac disease generally occurs in genetically susceptible individuals who possess either HLA-DQ2.5 (encoded by the genes HLA-DQA1*05 and HLA-DQB1*02) accounting for about 90% of individuals, HLA-DQ2.2 (encoded by the genes HLA-DQA1*02 and HLA-DQB1*02), or HLA-DQ8 (encoded by the genes HLA-DQA1*03 and HLA-DQB1*0302). Without wishing to be bound by theory, it is believed that such individuals mount an inappropriate HLA-DQ2- and/or DQ8-restricted CD4⁺ T cell-mediated immune response to peptides derived from aqueous-insoluble proteins of wheat flour, gluten, and related proteins in rye and barley.

### SUMMARY

Described herein are specific dosages and dosage schedules of a composition comprising at least one gluten peptide for use in treating subjects with Celiac disease. In some embodiments of any one of the methods provided, the composition comprises at least one peptide comprising at least one amino acid sequence selected from PFPQPELPY (SEQ ID NO: 4), PQPELPYPQ (SEQ ID NO: 5), PFPQPEQPF (SEQ ID NO: 6), PQPEQPFPW (SEQ ID NO: 7), PIPEQPQPY (SEQ ID NO: 8) and EQPIPEQPQ (SEQ ID NO: 9). In some embodiments of any one of the methods provided, the composition comprises at least one peptide selected from a first peptide comprising the amino acid sequence PFPQPELPY (SEQ ID NO: 4) and/or PQPELPYPQ; a second peptide comprising the amino acid sequence PFPQPEQPF and/or PQPEQPFPW; and a third peptide comprising the amino acid sequence PIPEQPQPY and/or EQPIPEQPQ. In some embodiments of any one of the methods provided, the composition comprises the first, second and third peptides. In some embodiments of any one of the methods provided, the composition comprises a first peptide comprising the amino acid sequence ELQPFPQPELPYPQPQ (SEQ ID NO: 1), wherein the N-terminal glutamate is a pyroglutamate and the carboxyl group of the C-terminal glutamine is amidated; a second peptide comprising the amino acid sequence EQPFPQPEQPFPWQP (SEQ ID NO: 2), wherein the N-terminal glutamate is a pyroglutamate and the carboxyl group of the C-terminal proline is amidated; and a third peptide comprising the amino acid sequence EPEQPIPEQPQPYPQQ (SEQ ID NO: 3), wherein the N-terminal glutamate is a pyroglutamate and the carboxyl group of the C-terminal glutamine is amidated.

Without being bound by theory, it is believed that administration of the compositions provided herein according to the dosages and dosage schedules described herein to a subject with Celiac disease can induce immune tolerance in the subject such that the subject may consume or come into contact with wheat, rye, and/or barley and, optionally, oats without a significant T cell response which would normally lead to symptoms of Celiac disease. In particular, in addition to a tolerizing dose period of the composition, a dose escalation period is contemplated prior to the tolerizing dose to gradually increase the dose administered to the subject (e.g., to reduce side effects).

Accordingly, aspects of the disclosure relate to compositions and methods for treating a subject with Celiac disease.

In some aspects, a method for treating Celiac disease in a subject is provided. In some embodiments, the method comprises administering to the subject one or more compositions comprising 0.9, 3, 9, 30, 60, 90, and/or 150 micrograms of one or more gluten peptides; and subsequently administering to the subject a second composition comprising 150 or 300 micrograms of one or more gluten peptides.

In some embodiments of any one of the methods provided, the method comprises administering to the subject a first composition comprising 0.9 micrograms of one or more gluten peptides, a third composition comprising 3 micrograms of one or more gluten peptides, a fourth composition comprising 9 micrograms of one or more gluten peptides, a fifth composition comprising 30 micrograms of one or more gluten peptides, a sixth composition comprising 90 micrograms of one or more gluten peptides, and subsequently administering to the subject a seventh composition comprising 150 micrograms of one or more gluten peptides.

In some embodiments, the method comprises administering to the subject one or more compositions comprising 3, 9, 30, 60, 90, and/or 150 micrograms of one or more gluten peptides; and subsequently administering to the subject a second composition comprising 300 micrograms of one or more gluten peptides.

In some embodiments of any one of the methods provided, the method comprises administering to the subject, such as one having a homozygous HLA-DQ2.5 genotype or a non-homozygous HLA-DQ2.5 genotype, a first composition comprising 3 micrograms of one or more gluten peptides, a third composition comprising 9 micrograms of one or more gluten peptides, a fourth composition comprising 30 micrograms of one or more gluten peptides, a fifth composition comprising 60 micrograms of one or more gluten peptides, a sixth composition comprising 90 micrograms of one or more gluten peptides, and subsequently administering to the subject a seventh composition comprising 150 micrograms of one or more gluten peptides.

In some embodiments of any one of the methods provided, the second composition comprises 50 micrograms of the first peptide and an equimolar amount of each of the second and third peptides. In some embodiments of any one of the methods provided, the second composition comprises 100 micrograms of the first peptide and an equimolar amount of each of the second and third peptides.

In some embodiments of any one of the methods provided, the second composition is administered six or eight times to the subject.

In some embodiments, the method comprises administering to the subject one or more compositions comprising 0.3, 1, 3, 10, 20, 30, and/or 50 micrograms of a first peptide and an equimolar amount of each of a second and a third peptide; and subsequently administering to the subject a second composition comprising 50 or 100 micrograms of the first peptide and an equimolar amount of each of the second and third peptides.

In some embodiments of any one of the methods provided,

(i) the first peptide comprises the amino acid sequence ELQPFPQPELPYPQPQ (SEQ ID NO: 1), wherein the N-terminal glutamate is a pyroglutamate and the C-terminal glutamine is amidated;

(ii) the second peptide comprises the amino acid sequence EQPFPQPEQPFPWQP (SEQ ID NO: 2), wherein the N-terminal glutamate is a pyroglutamate and the C-terminal proline is amidated; and

(iii) the third peptide comprises the amino acid sequence EPEQPIPEQPQPYPQQ (SEQ ID NO: 3), wherein the N-terminal glutamate is a pyroglutamate and the C-terminal glutamine is amidated.

In some embodiments of any one of the methods provided herein, each composition comprising one or more gluten peptides can comprise or consist of the aforementioned first, second, and third peptides. In some embodiments of any one of the methods provided, the first, second and third peptides are in equimolar amounts in each of compositions comprising one or more gluten peptides. In some embodiments of any one of the methods provided, each of the compositions comprising one or more gluten peptides are/is administered intradermally. In some embodiments of any one of the methods provided, the compositions comprising one or more gluten peptides are/is administered as a bolus by intradermal injection. In some embodiments of any one of the methods provided, each of the compositions comprising one or more gluten peptides are/is formulated as a sterile, injectable solution. In some embodiments of any one of the methods provided, the sterile, injectable solution is sodium chloride. In some embodiments of any one of the methods provided, the sodium chloride is sterile sodium chloride 0.9% USP.

In some embodiments of any one of the methods provided, the first composition comprises 0.3 micrograms of the first peptide and an equimolar amount of each of the second and third peptides, and the method further comprises administering to the subject a third composition comprising 1 microgram of the first peptide and an equimolar amount of each of the second and third peptides; administering to the subject a fourth composition comprising 3 micrograms of the first peptide and an equimolar amount of each of the second and third peptides; administering to the subject a fifth composition comprising 10 micrograms of the first peptide and an equimolar amount of each of the second and third peptides; and administering to the subject a sixth composition comprising 30 micrograms of the first peptide and an equimolar amount of each of the second and third peptides, wherein the third, fourth, fifth, and sixth composition are administered after administration of the first composition and before administration the second composition.

In some embodiments of any one of the methods provided, the first, third, fourth, fifth and sixth compositions are each administered once to the subject and the second composition is administered 16 times to the subject. In some embodiments of any one of the methods provided, the first, third, fourth, fifth and sixth compositions are administered over 2.5 weeks, with administrations occurring at a frequency of twice a week. In some embodiments of any one of the methods provided, the second composition is administered twice a week for 8 weeks.

In some embodiments of any one of the methods provided, the first composition comprises 1 microgram of the first peptide and an equimolar amount of each of the second and third peptides, and the method further comprises administering to the subject a third composition comprising 3 micrograms of the first peptide and an equimolar amount of each of the second and third peptides; and administering to the subject a fourth composition comprising 10 micrograms of the first peptide and an equimolar amount of each of the second and third peptides, administering to the subject a fifth composition comprising 20 micrograms of the first peptide and an equimolar amount of each of the second and third peptides, administering to the subject a sixth composition comprising 30 micrograms of the first peptide and an equimolar amount of each of the second and third peptides, wherein the third, fourth, fifth and sixth composition are administered after administration of the first composition and before administration the second composition. In some embodiments of any one of the methods provided, the first, third, fourth, fifth, and sixth composition are each administered once to the subject and the second composition is administered 8 times to the subject. In some embodiments of any one of the methods provided, the second composition is administered twice a week for 4 weeks.

In some embodiments of any one of the methods provided, the first, second and third peptides are in equimolar amounts in each of the first, second, third, fourth, fifth and/or sixth composition(s).

In some embodiments of any one of the methods provided, each of the first, second, third, fourth, fifth and/or sixth composition(s) are/is administered intradermally.

In some embodiments of any one of the methods provided, the first, second, third, fourth, fifth and/or sixth composition(s) are/is administered as a bolus by intradermal injection.

In some embodiments of any one of the methods provided, each of the first, second, third, fourth, fifth and/or sixth composition(s) are/is formulated as a sterile, injectable solution. In some embodiments, the sterile, injectable solution is sodium chloride. In some embodiments, the sodium chloride is sterile sodium chloride 0.9% USP.

In some embodiments of any one of the methods provided, the subject is HLA-DQ2.5 positive. In some embodiments of any one of the methods provided, the subject is on a gluten-free diet.

In some embodiments of any one of the methods provided, the method further comprises administering a composition comprising wheat, barley and/or rye (e.g., a composition comprising 6 grams of gluten) to the subject after the second composition is administered. In some embodiments of any one of the methods provided, the administration of the composition comprising wheat, barley and/or rye is for 6 weeks.

In some aspects, a method for treating Celiac disease in a subject, such as one having a non-homozygous HLA-DQ2.5 genotype, is provided. In some embodiments of any of the methods provided, the method comprises administering to the subject compositions comprising 3, 9, 30, 60, 90, 150, 300, 450, 600, 750 and/or 900 micrograms of one or more gluten peptides; and subsequently administering to the subject a second composition comprising 300 micrograms or up to 900 micrograms of one or more gluten peptides.

In some embodiments of any one of the methods provided, the method comprises administering to the subject, such as one having a non-homozygous HLA-DQ2.5 genotype, a first composition comprising 30 micrograms of one or more gluten peptides, a third composition comprising 60 micrograms of one or more gluten peptides, a fourth composition comprising 90 micrograms of one or more gluten peptides, a fifth composition comprising 150 micrograms of one or more gluten peptides, a sixth composition comprising 300 micrograms of one or more gluten peptides, a seventh composition comprising 450 micrograms of one or more gluten peptides, an eighth composition comprising 600 micrograms of one or more gluten peptides, a ninth composition comprising 750 micrograms of one or more gluten peptides and subsequently administering to the subject a tenth composition comprising 900 micrograms of one or more gluten peptides, wherein the third, fourth, fifth, sixth, seventh, eighth, ninth, and tenth compositions are administered after administration of the first composition and before administration the second composition.

In some embodiments of any of the methods provided, the method comprises administering to the subject compositions comprising 3, 9, 30, 60, 90, 150, 300, 450, 600 and/or 750 micrograms of one or more gluten peptides; and subsequently administering to the subject a second composition comprising 300 micrograms or up to 900 micrograms of one or more gluten peptides.

In some embodiments of any one of the methods provided herein, the method comprises administering to the subject, such as one having a non-homozygous HLA-DQ2.5 genotype, a first composition comprising 3 micrograms of one or more gluten peptides, a third composition comprising 9 micrograms of one or more gluten peptides, a fourth composition comprising 30 micrograms of one or more gluten peptides, a fifth composition comprising 60 micrograms of one or more gluten peptides, a sixth composition comprising 90 micrograms of one or more gluten peptides, a seventh composition comprising 150 micrograms of one or more gluten peptides, an eighth composition comprising 300 micrograms of one or more gluten peptides, a ninth composition comprising 450 micrograms of one or more gluten peptides, a tenth composition comprising 600 micrograms of one or more gluten peptides, and subsequently administering to the subject an eleventh composition comprising 750 micrograms of one or more gluten peptides, wherein the third, fourth, fifth, sixth, seventh, eighth, ninth, tenth, and eleventh compositions are administered after administration of the first composition and before administration the second composition.

In some embodiments of any one of the methods provided, the second composition comprises 300 micrograms of one or more gluten peptides. In some embodiments of any one of the methods provided, the second composition comprises up to 900 micrograms (e.g., 900 micrograms) of one or more gluten peptides.

In some embodiments of any one of the methods provided, the non-homozygous HLA-DQ2.5 genotype is a heterozygous HLA-DQ2.5 genotype. In some embodiments of any one of the methods provided, the heterozygous HLA-DQ2.5 genotype is HLA-DQ2.5/2.2, HLA-DQ2.5/7, or HLA-DQ2.5/8.

In some embodiments of any one of the methods provided, the first, third, fourth, fifth sixth, seventh, eighth, ninth, and tenth compositions are administered over 4.5 weeks, with administrations occurring at a frequency of twice a week. In some embodiments of any one of the methods provided, the second composition is administered twice a week for 4 weeks.

In some embodiments of any one of the methods provided, the first, third, fourth, fifth sixth, seventh, eighth, ninth, tenth, and eleventh compositions are administered over 4.5 weeks, with administrations occurring at a frequency of twice a week. In some embodiments of any one of the methods provided, the second composition is administered twice a week for 4 weeks.

In some embodiments of any one of the methods provided, the second composition is administered six or eight times to the subject.

Also provided herein in an aspect is a method of treating a subject with Celiac disease, the method comprising any one of the titration phases provided herein, comprising any one of the tolerizing phases provided herein, or both any one of the titration phases and any one of the tolerizing phases provided herein.

### BRIEF DESCRIPTION OF THE DRAWING

The following drawings form part of the present specification and are included to further demonstrate certain aspects of the present disclosure, which can be better understood by reference to one or more of these drawings in combination with the detailed description of specific embodiments presented herein.

FIG. 1 is an exemplary schematic of a study design to evaluate dose titration and push dose.

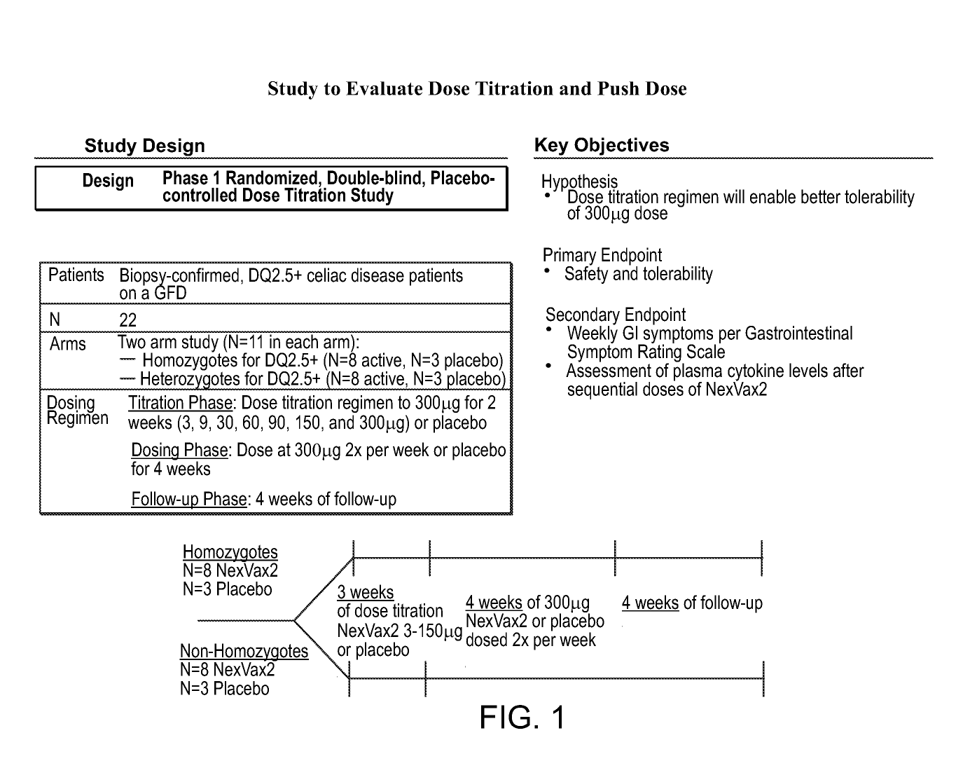

FIG. 2 is an exemplary schematic of a study design to evaluate dose titration.

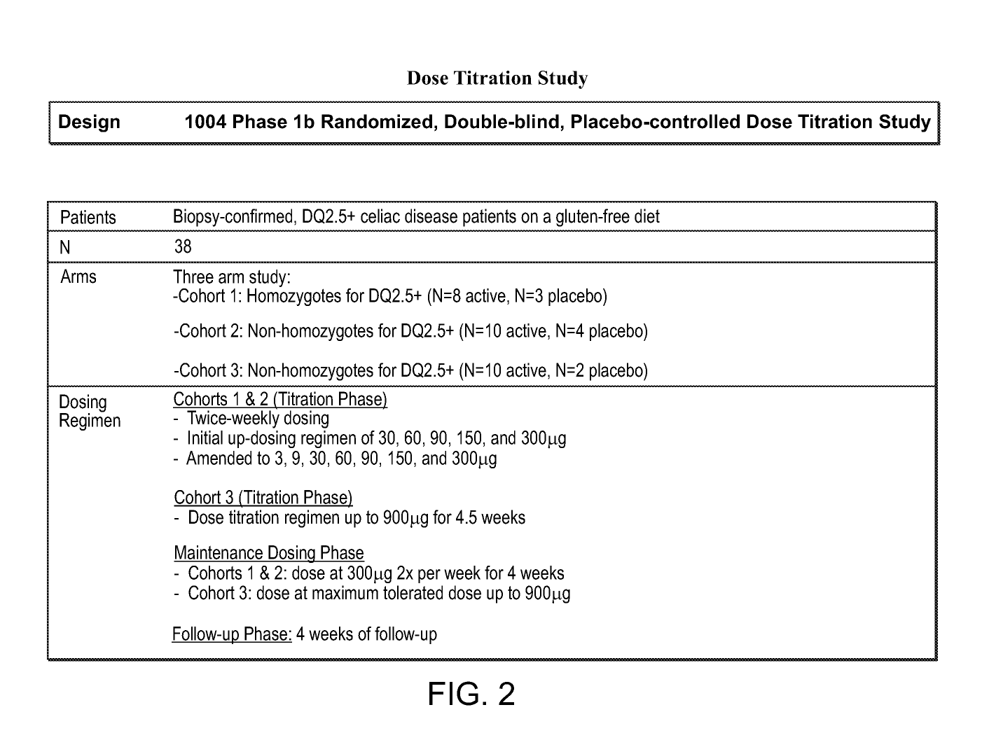

FIG. 3 is a graph showing dosage numbers and dosage amounts (micrograms) in dosage administration studies. Incorporation of an up-dosing regimen enabled patients to achieve and maintain 6 times higher dose versus a fixed-dose regimen.

FIG. 4 is a series of graphs depicting plasma concentrations of gluten peptide compositions before and after dosing.

FIG. 5 is a graph depicting incidence and severity of adverse events in subjects receiving an up-dosing regimen of gluten peptide composition.

FIG. 6 is a graph depicting IL-2 level in subjects receiving an up-dosing regimen of gluten peptide composition.

FIG. 7 is a series of graphs depicting IL-2 release in plasma in fixed dosing (left and middle panels) and up-dosing (right panel) regimens.

FIG. 8 is a graph depicting Gastrointestinal Symptom Rating Scale (GSRS) score over time (lower numbers indicate lesser symptom severity). Overall symptom scores were measured at baseline and then weekly. Placebo patients pooled all cohorts. Updosing begins at 3 micrograms and the top dose was 900 micrograms. A significant reduction in symptoms compared to baseline was seen. No difference in symptoms between baseline and treatment period was seen in the placebo group.

FIG. 9 is a table summarizing the weekly GI symptom diary across treatment period related to pain or discomfort.

FIG. 10 is a table summarizing the weekly GI symptom diary across treatment period related to nausea.

FIG. 11 is a graph depicting no difference in duodenal morphometry in Cohort 3 (n=10 CeD patients).

### DETAILED DESCRIPTION

### General Techniques and Definitions

Unless specifically defined otherwise, all technical and scientific terms used herein shall be taken to have the same meaning as commonly understood by one of ordinary skill in the art (e.g., in cell culture, molecular genetics, immunology, immunohistochemistry, protein chemistry, and biochemistry).

Unless otherwise indicated, techniques utilized in the present disclosure are standard procedures, well known to those skilled in the art. Such techniques are described and explained throughout the literature in sources such as, J. Sambrook et al., Molecular Cloning: A Laboratory Manual, Cold Spring Harbour Laboratory Press (2012); T. A. Brown (editor), Essential Molecular Biology: A Practical Approach, Volumes 1 and 2, IRL Press (2000 and 2002); D. M. Glover and B. D. Hames (editors), Current Protocols in Molecular Biology, Greene Pub. Associates and Wiley-Interscience (1988, including all updates until present); Edward A. Greenfield (editor) Antibodies: A Laboratory Manual, Cold Spring Harbour Laboratory, (2013); and J. E. Coligan et al. (editors), Current Protocols in Immunology, John Wiley & Sons (including all updates until present).

The term “Celiac disease” generally refers to an immune-mediated systemic disorder elicited by gluten and related prolamines in genetically susceptible individuals, characterized by the presence of a variable combination of gluten-dependent clinical manifestations, celiac disease-specific antibodies, human leukocyte antigen (HLA)-DQ2 and HLA-DQ8 haplotypes, and enteropathy. The disease encompasses a spectrum of conditions characterised by an inappropriate CD4⁺ T cell response to gluten, or a peptide thereof. The severe form of celiac disease is characterised by a flat small intestinal mucosa (hyperplastic villous atrophy) and other forms are characterised by milder histological abnormalities in the small intestine, such as intra-epithelial lymphocytosis without villous atrophy. Serological abnormalities associated with celiac disease generally include the presence of autoantibodies specific for tissue transglutaminase-2 and antibodies specific for deamidated gluten-derived peptides. The clinical manifestations associated with celiac disease can include fatigue, chronic diarrhoea, malabsorption of nutrients, weight loss, abdominal distension, anaemia as well as a substantially enhanced risk for the development of osteoporosis and intestinal malignancies (lymphoma and carcinoma).

The terms “human leukocyte antigen” and “HLA” are here defined as a genetic fingerprint expressed on human white blood cells, composed of proteins that play a critical role in activating the body's immune system to respond to foreign organisms. In humans and other animals, the HLA is also collectively referred to as the “major histocompatibility complex” (MHC).

The term “subject” includes inter alia an individual, patient, target, host or recipient regardless of whether the subject is a human or non-human animal including mammalian species and also avian species. The term “subject”, therefore, includes a human, non-human primate (for example, gorilla, marmoset, African Green Monkey), livestock animal (for example, sheep, cow, pig, horse, donkey, goat), laboratory test animal (for example, rat, mouse, rabbit, guinea pig, hamster), companion animal (for example, dog, cat), captive wild animal (for example, fox, deer, game animals) and avian species including poultry birds (for example, chickens, ducks, geese, turkeys). The preferred subject, however, is a human. In some embodiments, the subject is a human on a gluten-free diet. In some embodiments, the subject is a human who is HLA-DQ2.5 positive. In some embodiments, the subject is a human who is HLA-DQ2.5 positive and HLA-DQ8 negative. In some embodiments, the subject is a human who is HLA-DQ2.5 positive and HLA-DQ8 positive.

### Peptides

The terms “peptide”, “polypeptide”, and “protein” can generally be used interchangeably and also encompass pharmaceutical salts thereof. In some embodiments of any one of the methods or compositions provided herein, the term “peptide” is used to refer to relatively short molecules comprising less than 50, more preferably less than 25, amino acids.

The overall length of each peptide defined herein may be, for example, 7 to 50 amino acids, such as 7, 8, 9 10, 11, 12, 13, 14, 15, 16, 17, 18, 19, 20, 21, 22, 23, 24, 25, 30, 35, 40, 45, or 50 amino acids, or any integer in between. It is contemplated that shorter peptides may prove useful, particularly those that are 20 or fewer amino acids in length, in therapeutics to reduce the likelihood of anaphylaxis but longer peptides with multiple epitopes are likely to be as effective as multiple short peptides, for example, in functional T cell-based diagnostics in vitro.

It is believed that the peptides of the disclosure, such as those that comprise SEQ ID NOs: 1, 2, and 3, are capable of generating a T cell response in a subject having Celiac disease. Without wishing to be bound by theory, T cell responses in a subject with Celiac disease can be caused by T-cell receptor ligation of the minimal T cell epitopes present in SEQ ID NOs: 1, 2, and 3 that are presented by HLA-DQ2.5 on the surface of antigen presenting cells.

In some embodiments, a peptide is modified during or after translation or synthesis (for example, by farnesylation, prenylation, myristoylation, glycosylation, palmitoylation, acetylation, phosphorylation [such as phosphotyro sine, phosphoserine or phosphothreonine], amidation, derivatisation by known protecting/blocking groups, proteolytic cleavage, linkage to an antibody molecule or other cellular ligand, and the like). Any of the numerous chemical modification methods known within the art may be utilised including, but not limited to, specific chemical cleavage by cyanogen bromide, trypsin, chymotrypsin, papain, V8 protease, NaB H₄, acetylation, formylation, oxidation, reduction, metabolic synthesis in the presence of tunicamycin, etc.

The phrases “protecting group” and “blocking group” as used herein, refers to modifications to the peptide, which protect it from undesirable chemical reactions, particularly in vivo. Examples of such protecting groups include esters of carboxylic acids and boronic acids, ethers of alcohols and acetals, and ketals of aldehydes and ketones. Examples of suitable groups include acyl protecting groups such as, for example, furoyl, formyl, adipyl, azelayl, suberyl, dansyl, acetyl, theyl, benzoyl, trifluoroacetyl, succinyl and methoxysuccinyl; aromatic urethane protecting groups such as, for example, benzyloxycarbonyl (Cbz); aliphatic urethane protecting groups such as, for example, t-butoxycarbonyl (Boc) or 9-fluorenylmethoxy-carbonyl (FMOC); pyroglutamate and amidation. Many other modifications providing increased potency, prolonged activity, ease of purification, and/ or increased half-life will be known to the person skilled in the art.

The peptides may comprise one or more modifications, which may be natural post-translation modifications or artificial modifications. The modification may provide a chemical moiety (typically by substitution of a hydrogen, for example, of a C—H bond), such as an amino, acetyl, acyl, amide, carboxy, hydroxy or halogen (for example, fluorine) group, or a carbohydrate group. Typically, the modification is present on the N- and/or C-terminus. Furthermore, one or more of the peptides may be PEGylated, where the PEG (polyethyleneoxy group) provides for enhanced lifetime in the blood stream. One or more of the peptides may also be combined as a fusion or chimeric protein with other proteins, or with specific binding agents that allow targeting to specific moieties on a target cell. The peptide may also be chemically modified at the level of amino acid side chains, of amino acid chirality, and/ or of the peptide backbone.

Particular changes can be made to the peptides to improve resistance to degradation or optimise solubility properties or otherwise improve bioavailability compared to the parent peptide, thereby providing peptides having similar or improved therapeutic, diagnostic and/or pharmacokinetic properties. A preferred such modification includes the use of an N-terminal pyroglutamate and/ or a C-terminal amide (such as the respective N-terminal pyroglutamate and C-terminal glutamine of SEQ ID NOs: 1, 2, and 3). Such modifications have been shown previously to significantly increase the half-life and bioavailability of the peptides compared to the parent peptides having a free N- and C-terminus.

In a particular embodiment, a composition comprising a first peptide comprising the amino acid sequence ELQPFPQPELPYPQPQ (SEQ ID NO: 1), wherein the N-terminal glutamate is a pyroglutamate and the C-terminal glutamine is amidated (i.e., the free C-terminal COO is amidated); a second peptide comprising the amino acid sequence EQPFPQPEQPFPWQP (SEQ ID NO: 2), wherein the N-terminal glutamate is a pyroglutamate and the C-terminal proline is amidated (i.e., the free C-terminal COO is amidated); and a third peptide comprising the amino acid sequence EPEQPIPEQPQPYPQQ (SEQ ID NO: 3), wherein the N-terminal glutamate is a pyroglutamate and the C-terminal glutamine is amidated (i.e., the free C-terminal COO is amidated) is contemplated. In some embodiments, the first, second and/or third peptides consist essentially of or consist of the amino acid sequence of SEQ ID NO: 1, 2, or 3, respectively. Compositions are further described herein.

Certain peptides described herein may exist in particular geometric or stereoisomeric forms. The present disclosure contemplates all such forms, including cis- (Z) and trans-(E) isomers, R- and S-enantiomers, diastereomers, (D)-isomers, (L)-isomers, the racemic mixtures thereof, and other mixtures thereof, as, falling within the scope of the disclosure. Additional asymmetric carbon atoms may be present in a substituent, such as an alkyl group. All such isomers, as well as mixtures thereof, are intended to be included in this disclosure. In another example, to prevent cleavage by peptidases, any one or more of the peptides may include a non-cleavable peptide bond in place of a particularly sensitive peptide bond to provide a more stable peptide. Such non cleavable peptide bonds may include beta amino acids.

In certain embodiments, any one or more of the peptides may include a functional group, for example, in place of the scissile peptide bond, which facilitates inhibition of a serine-, cysteine- or aspartate-type protease, as appropriate. For example, the disclosure includes a peptidyl diketone or a peptidyl keto ester, a peptide haloalkylketone, a peptide sulfonyl fluoride, a peptidyl boronate, a peptide epoxide, a peptidyl diazomethane, a peptidyl phosphonate, isocoumarins, benzoxazin-4-ones, carbamates, isocyantes, isatoic anhydrides or the like. Such functional groups have been provided in other peptide molecules, and general routes for their synthesis are known.

The peptides may be in a salt form, preferably, a pharmaceutically acceptable salt form. “A pharmaceutically acceptable salt form” includes the conventional non-toxic salts or quaternary ammonium salts of a peptide, for example, from non-toxic organic or inorganic acids. Conventional non-toxic salts include, for example, those derived from inorganic acids such as hydrochloride, hydrobromic, sulphuric, sulfonic, phosphoric, nitric, and the like; and the salts prepared from organic acids such as acetic, propionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, ethane disulfonic, oxalic, isothionic, and the like.

### Peptide Production

The peptides can be prepared in any suitable manner. For example, the peptides can be recombinantly and/or synthetically produced.

The peptides may be synthesised by standard chemistry techniques, including synthesis by an automated procedure using a commercially available peptide synthesiser. In general, peptides may be prepared by solid-phase peptide synthesis methodologies which may involve coupling each protected amino acid residue to a resin support, preferably a 4-methylbenzhydrylamine resin, by activation with dicyclohexylcarbodiimide to yield a peptide with a C-terminal amide. Alternatively, a chloromethyl resin (Merrifield resin) may be used to yield a peptide with a free carboxylic acid at the C-terminal. After the last residue has been attached, the protected peptide-resin is treated with hydrogen fluoride to cleave the peptide from the resin, as well as deprotect the side chain functional groups. Crude product can be further purified by gel filtration, high pressure liquid chromatography (HPLC), partition chromatography, or ion-exchange chromatography.

If desired, and as outlined above, various groups may be introduced into the peptide of the composition during synthesis or during expression, which allow for linking to other molecules or to a surface. For example, cysteines can be used to make thioethers, histidines for linking to a metal ion complex, carboxyl groups for forming amides or esters, amino groups for forming amides, and the like.

The peptides may also be produced using cell-free translation systems. Standard translation systems, such as reticulocyte lysates and wheat germ extracts, use RNA as a template; whereas “coupled” and “linked” systems start with DNA templates, which are transcribed into RNA then translated.

Alternatively, the peptides may be produced by transfecting host cells with expression vectors that comprise a polynucleotide(s) that encodes one or more peptides. For recombinant production, a recombinant construct comprising a sequence which encodes one or more of the peptides is introduced into host cells by conventional methods such as calcium phosphate transfection, DEAE-dextran mediated transfection, microinjection, cationic lipid-mediated transfection, electroporation, transduction, scrape lading, ballistic introduction or infection.

One or more of the peptides may be expressed in suitable host cells, such as, for example, mammalian cells (for example, COS, CHO, BHK, 293 HEK, VERO, HeLa, HepG2, MDCK, W138, or NIH 3T3 cells), yeast (for example, Saccharomyces or Pichia), bacteria (for example, E. coli, P. pastoris, or B. subtilis), insect cells (for example, baculovirus in Sf9 cells) or other cells under the control of appropriate promoters using conventional techniques. Following transformation of the suitable host strain and growth of the host strain to an appropriate cell density, the cells are harvested by centrifugation, disrupted by physical or chemical means, and the resulting crude extract retained for further purification of the peptide or variant thereof.

Suitable expression vectors include, for example, chromosomal, non-chromosomal and synthetic polynucleotides, for example, derivatives of SV40, bacterial plasmids, phage DNAs, yeast plasmids, vectors derived from combinations of plasmids and phage DNAs, viral DNA such as vaccinia viruses, adenovirus, adeno-associated virus, lentivirus, canary pox virus, fowl pox virus, pseudorabies, baculovirus, herpes virus and retrovirus. The polynucleotide may be introduced into the expression vector by conventional procedures known in the art.

The polynucleotide which encodes one or more peptides may be operatively linked to an expression control sequence, i.e., a promoter, which directs mRNA synthesis. Representative examples of such promoters include the LTR or SV40 promoter, the E. coli lac or trp, the phage lambda PL promoter and other promoters known to control expression of genes in prokaryotic or eukaryotic cells or in viruses. The expression vector may also contain a ribosome binding site for translation initiation and a transcription terminator. The expression vectors may also include an origin of replication and a selectable marker, such as the ampicillin resistance gene of E. coli to permit selection of transformed cells, i.e., cells that are expressing the heterologous polynucleotide. The nucleic acid molecule encoding one or more of the peptides may be incorporated into the vector in frame with translation initiation and termination sequences.

One or more of the peptides can be recovered and purified from recombinant cell cultures (i.e., from the cells or culture medium) by well-known methods including ammonium sulphate or ethanol precipitation, acid extraction, anion or cation exchange chromatography, phosphocellulose chromatography, hydrophobic interaction chromatography, affinity chromatography, hydroxyapatite chromatography, lectin chromatography, and HPLC. Well known techniques for refolding proteins may be employed to regenerate active conformation when the peptide is denatured during isolation and or purification.

To produce a glycosylated peptide, it is preferred that recombinant techniques be used. To produce a glycosylated peptide, it is preferred that mammalian cells such as, COS-7 and Hep-G2 cells be employed in the recombinant techniques.

The peptides can also be prepared by cleavage of longer peptides, especially from food extracts.

Pharmaceutically acceptable salts of the peptides can be synthesised from the peptides which contain a basic or acid moiety by conventional chemical methods. Generally, the salts are prepared by reacting the free base or acid with stoichiometric amounts or with an excess of the desired salt-forming inorganic or organic acid or base in a suitable solvent.

### Gluten Challenge

In some embodiments, any one of the methods provided herein comprises a gluten challenge or a sample obtained from a subject before, during, or after a gluten challenge. Generally, a gluten challenge comprises administering to the subject a composition comprising wheat, rye, or barley, or one or more peptides thereof (e.g., a composition comprising a wheat gliadin, a rye secalin, or a barley hordein, or one or more peptides thereof), in some form for a defined period of time in order to activate the immune system of the subject, e.g., through activation of wheat-, rye- and/or barley-reactive T cells and/or mobilization of such T cells in the subject. Methods of gluten challenges are well known in the art and include oral, submucosal, supramucosal, and rectal administration of peptides or proteins (see, e.g., Can J Gastroenterol. 2001. 15(4):243-7. In vivo gluten challenge in celiac disease. Ellis H J, Ciclitira P J; Mol Diagn Ther. 2008. 12(5):289-98. Celiac disease: risk assessment, diagnosis, and monitoring. Setty M, Hormaza L, Guandalini S; Gastroenterology. 2009;137(6):1912-33. Celiac disease: from pathogenesis to novel therapies. Schuppan D, Junker Y, Barisani D; J Dent Res. 2008;87(12):1100-1107. Orally based diagnosis of celiac disease: current perspectives. Pastore L, Campisi G, Compilato D, and Lo Muzio L; Gastroenterology. 2001;120:636-651. Current Approaches to Diagnosis and Treatment of Celiac Disease: An Evolving Spectrum. Fasano A and Catassi C; Clin Exp Immunol. 2000;120:38-45. Local challenge of oral mucosa with gliadin in patients with coeliac disease. Lahteenoja M, Maki M, Viander M, Toivanen A, Syrjanen S; Clin Exp Immunol. 2000;120:10-11. The mouth-an accessible region for gluten challenge. Ellis H and Ciclitira P; Clinical Science. 2001;101:199-207. Diagnosing coeliac disease by rectal gluten challenge: a prospective study based on immunopathology, computerized image analysis and logistic regression analysis. Ensari A, Marsh M, Morgan S, Lobley R, Unsworth D, Kounali D, Crowe P, Paisley J, Moriarty K, and Lowry J; Gut. 2005;54:1217-1223. T cells in peripheral blood after gluten challenge in coeliac disease. Anderson R, van Heel D, Tye-Din J, Barnardo M, Salio M, Jewell D, and Hill A; and Nature Medicine. 2000;6(3):337-342. In vivo antigen challenge in celiac disease identifies a single transglutaminase-modified peptide as the dominant A-gliadin T-cell epitope. Anderson R, Degano P, Godkin A, Jewell D, and Hill A). Traditionally, a challenge lasts for several weeks (e.g., 4 weeks or more) and involves high doses of orally administered peptides or proteins (usually in the form of baked foodstuff that includes the peptides or proteins). Some studies suggest that a shorter challenge, e.g., through use of as little as 3 days of oral challenge, is sufficient to activate and/or mobilize reactive T-cells (Anderson R, van Heel D, Tye-Din J, Barnardo M, Salio M, Jewell D, and Hill A; and Nature Medicine. 2000;6(3):337-342. In vivo antigen challenge in celiac disease identifies a single transglutaminase-modified peptide as the dominant A-gliadin T-cell epitope. Anderson R, Degano P, Godkin A, Jewell D, and Hill A). In some embodiments, any one of the methods provided herein comprises performing a gluten challenge on the subject or obtaining a sample from a subject before, during or after a gluten challenge, where the gluten challenge is for 6 weeks. In some embodiments, a gluten escalation (e.g., administering increasing amounts of gluten over time to a subject) is performed before the gluten challenge.

In some embodiments of any one of the methods provided herein, the challenge comprises administering a composition comprising wheat, barley and/or rye, or one or more peptides thereof. In some embodiments, the wheat is wheat flour, the barely is barley flour, and the rye is rye flour. In some embodiments, the challenge comprises administering a composition comprising a wheat gliadin, a barley hordein and/or a rye secalin, or one or more peptides thereof, to the subject prior to determining a T cell response as described herein.

In some embodiments of any one of the methods provided herein, the composition is administered to the subject after administration of a dose escalation regimen and a tolerizing regimen as described herein. In some embodiments, a sample is obtained from the subject after administration of the composition. In some embodiments, administration is for 6 weeks. In some embodiments, the composition contains 6 grams of gluten.

In some embodiments, administration is oral. Suitable forms of oral administration include foodstuffs (e.g., baked goods such as breads, cookies, cakes, etc.), tablets, troches, lozenges, aqueous or oily suspensions, dispersible powders or granules, emulsions, hard or soft capsules, or syrups or elixirs. Compositions intended for oral use may be prepared according to methods known to the art for the manufacture of pharmaceutical compositions or foodstuffs and such compositions may contain one or more agents including, for example, sweetening agents, flavoring agents, coloring agents and preserving agents in order to provide pharmaceutically elegant and palatable preparations.

In some embodiments, a sample is obtained from a subject before, during, and/or after a gluten challenge as described herein.

### Compositions, Vaccine Compositions, and Administration

### Compositions and Vaccine Compositions

The disclosure also provides a composition comprising at least one gluten peptide as provided herein. In some embodiments of any one of the compositions or methods provided, the composition comprises at least one peptide comprising at least one amino acid sequence selected from PFPQPELPY (SEQ ID NO: 4), PQPELPYPQ (SEQ ID NO: 5), PFPQPEQPF (SEQ ID NO: 6), PQPEQPFPW (SEQ ID NO: 7), PIPEQPQPY (SEQ ID NO: 8) and EQPIPEQPQ (SEQ ID NO: 9). In some embodiments of any one of the compositions or methods provided, the composition comprises at least one peptide selected from a first peptide comprising the amino acid sequence PFPQPELPY (SEQ ID NO: 4) and/or PQPELPYPQ (SEQ ID NO: 5); a second peptide comprising the amino acid sequence PFPQPEQPF (SEQ ID NO: 6) and/or PQPEQPFPW (SEQ ID NO: 7); and a third peptide comprising the amino acid sequence PIPEQPQPY (SEQ ID NO: 8) and/or EQPIPEQPQ (SEQ ID NO: 9). In some embodiments, the composition comprises a first peptide comprising the amino acid sequence ELQPFPQPELPYPQPQ (SEQ ID NO: 1), wherein the N-terminal glutamate is a pyroglutamate and the carboxyl group of the C-terminal glutamine is amidated; a second peptide comprising the amino acid sequence EQPFPQPEQPFPWQP (SEQ ID NO: 2), wherein the N-terminal glutamate is a pyroglutamate and the carboxyl group of the C-terminal proline is amidated; and a third peptide comprising the amino acid sequence EPEQPIPEQPQPYPQQ (SEQ ID NO: 3), wherein the N-terminal glutamate is a pyroglutamate and the carboxyl group of the C-terminal glutamine is amidated. In some embodiments, the composition is a vaccine composition.

As used herein, the term “vaccine” refers to a composition comprising one or more peptides that can be administered to a subject having Celiac disease to modulate the subject's response to gluten. The vaccine may reduce the immunological reactivity of a subject towards gluten. Preferably, the vaccine induces tolerance to gluten.

Without being bound by any theory, administration of the vaccine composition to a subject may induce tolerance by clonal deletion of gluten-specific effector T cell populations, for example, gluten-specific CD4⁺ T cells, or by inactivation (anergy) of said T cells such that they become less responsive, preferably, unresponsive to subsequent exposure to gluten (or peptides thereof). Assessing immune tolerance, e.g., deletion or inactivation of said T cells can be measured, for example, by contacting ex vivo a sample comprising said T cells with gluten or a peptide thereof and measuring the response of said T cells to the gluten or peptide thereof. T cell response assays are known in the art (see, e.g., PCT Publication Number WO2010/060155).

Alternatively, or in addition, administration of the vaccine composition may modify the cytokine secretion profile of the subject (for example, result in decreased IL-4, IL-2, TNF-α and/or IFN-γ, and/or increased IL-10). The vaccine composition may induce suppressor T cell subpopulations, for example Treg cells, to produce IL-10 and/or TGF-β and thereby suppress gluten-specific effector T cells. The cytokine secretion profile of the subject can be measured using any method known to those of skill in the art, e.g., using immuno-based detection methods such as Western blot or enzyme-linked immunosorbent assay (ELISA).

The vaccine composition of the disclosure can be used for prophylactic treatment of a subject capable of developing Celiac disease and/or used in ongoing treatment of a subject who has Celiac disease. In some embodiments, the composition is for use in treating Celiac disease in a subject. In some embodiments, the subject is HLA-DQ2.5 positive. In some embodiments, the subject is HLA-DQ2.5 positive and HLA-DQ8 negative.

### Effective Amount

Compositions are generally administered in “effective amounts”. The term “effective amount” means the amount sufficient to provide the desired therapeutic or physiological effect when administered under appropriate or sufficient conditions. In some embodiments, the effective amount is 150 micrograms of the peptides provided herein (i.e., 50 micrograms of the first peptide and an equimolar amount of each of the second and third peptides). In some embodiments, the effective amount is 26.5 nmol of each of the first, second, and third peptides. In some embodiments, the effective amount is 300 micrograms of the peptides provided herein (i.e., 100 micrograms of the first peptide and an equimolar amount of each of the second and third peptides). In some embodiments, the effective amount is 0.9 micrograms of the peptides provided herein (i.e., 0.3 micrograms of the first peptide and an equimolar amount of each of the second and third peptides) or an equivalent, such as a molar equivalent, thereof. In some embodiments, the effective amount is 3 micrograms of the peptides provided herein (i.e., 1 micrograms of the first peptide and an equimolar amount of each of the second and third peptides) or an equivalent, such as a molar equivalent, thereof. In some embodiments, the effective amount is 9 micrograms of the peptides provided herein (i.e., 3 micrograms of the first peptide and an equimolar amount of each of the second and third peptides) or an equivalent, such as a molar equivalent, thereof. In some embodiments, the effective amount is 30 micrograms of the peptides provided herein (i.e., 10 micrograms of the first peptide and an equimolar amount of each of the second and third peptides) or an equivalent, such as a molar equivalent, thereof. In some embodiments, the effective amount is 60 micrograms of the peptides provided herein (i.e., 20 micrograms of the first peptide and an equimolar amount of each of the second and third peptides) or an equivalent, such as a molar equivalent, thereof. In some embodiments, the effective amount is 90 micrograms of the peptides provided herein (i.e., 30 micrograms of the first peptide and an equimolar amount of each of the second and third peptides) or an equivalent, such as a molar equivalent, thereof. In some embodiments, the effective amount is 150 micrograms of the peptides provided herein (i.e., 50 micrograms of the first peptide and an equimolar amount of each of the second and third peptides) or an equivalent, such as a molar equivalent, thereof.

Methods for producing equimolar peptide compositions are known in the art and provided herein (see, e.g., Example 1 and Muller et al. Successful immunotherapy with T-cell epitope peptides of bee venom phospholipase A2 induces specific T-cell anergy in patient allergic to bee venom. J. Allergy Clin. Immunol. Vol. 101, Number 6, Part 1:747-754 (1998)). In some embodiments, multiple effective dosages are utilized, e.g., to provide dose escalation. In some embodiments, one or more effective amounts of the peptides are administered in sterile sodium chloride 0.9% USP as a bolus intradermal injection.

The effective amounts provided herein, when used alone or in combination as part of a dosage schedule, are believed to modify the T cell response, e.g., by inducing immune tolerance, to wheat, barley and rye in the subject, and preferably wheat, barley, rye and oats. Thus, a subject treated according to the disclosure preferably is able to eat at least wheat, rye, barley and, optionally, oats without a significant T cell response which would normally lead to clinical manifestations of active Celiac disease.

### Pharmaceutically Acceptable Carriers

The compositions provided herein may include a pharmaceutically acceptable carrier. The term “pharmaceutically acceptable carrier” refers to molecular entities and compositions that do not produce an allergic, toxic or otherwise adverse reaction when administered to a subject, particularly a mammal, and more particularly a human. The pharmaceutically acceptable carrier may be solid or liquid. Useful examples of pharmaceutically acceptable carriers include, but are not limited to, diluents, excipients, solvents, surfactants, suspending agents, buffering agents, lubricating agents, adjuvants, vehicles, emulsifiers, absorbants, dispersion media, coatings, stabilizers, protective colloids, adhesives, thickeners, thixotropic agents, penetration agents, sequestering agents, isotonic and absorption delaying agents that do not affect the activity of the active agents of the disclosure. In some embodiments, the pharmaceutically acceptable carrier is a sodium chloride solution (e.g., sodium chloride 0.9% USP).

The carrier can be any of those conventionally used and is limited only by chemico-physical considerations, such as solubility and lack of reactivity with the active agent, and by the route of administration. Suitable carriers for this disclosure include those conventionally used, for example, water, saline, aqueous dextrose, lactose, Ringer's solution, a buffered solution, hyaluronan, glycols, starch, cellulose, glucose, lactose, sucrose, gelatin, malt, rice, flour, chalk, silica gel, magnesium stearate, sodium stearate, glycerol monostearate, sodium chloride, glycerol, propylene glycol, water, ethanol, and the like. Liposomes may also be used as carriers.

Techniques for preparing pharmaceutical compositions are generally known in the art as exemplified by Remington's Pharmaceutical Sciences, 16th Ed. Mack Publishing Company, 1980.

Administration preferably is intradermal administration. Thus, the composition(s) of the disclosure may be in a form suitable for intradermal injection. In some embodiments, the composition(s) of the disclosure are in the form of a bolus for intradermal injection.

### Injectables

The pharmaceutical composition(s) may be in the form of a sterile injectable aqueous or oleagenous suspension. In some embodiments, the composition is formulated as a sterile, injectable solution. This suspension or solution may be formulated according to known methods using those suitable dispersing or wetting agents and suspending agents which have been mentioned above. The sterile injectable preparation may be a suspension in a non-toxic parenterally-acceptable diluent or solvent, for example as a solution in 1,3-butanediol. Among the acceptable carriers that may be employed are water, Ringer's solution and isotonic sodium chloride solution. In some embodiments, the composition is formulated as a sterile, injectable solution, wherein the solution is a sodium chloride solution (e.g., sodium chloride 0.9% USP). In some embodiments, the composition is formulated as a bolus for intradermal injection.

Examples of appropriate delivery mechanisms for intradermal administration include, but are not limited to, implants, depots, needles, capsules, and osmotic pumps.

### Dosage

It is especially advantageous to formulate the active in a dosage unit form for ease of administration and uniformity of dosage. “Dosage unit form” as used herein refers to physically discrete units suited as unitary dosages for the subject to be treated; each unit containing a predetermined quantity of active agent calculated to produce the desired therapeutic effect in association with a pharmaceutical carrier. The specification for the dosage unit forms are dictated by and directly dependent on the unique characteristics of the active agent and the particular therapeutic effect to be achieved, and the limitations inherent in the art of compounding such an active agent for the treatment of subjects. Examples of dosage units include sealed ampoules and vials and may be stored in a freeze-dried condition requiring only the addition of the sterile liquid carrier immediately prior to use.

The composition(s) may also be included in a container, pack, or dispenser together with instructions for administration.

The actual amount(s) administered (or dose or dosage) and the rate and time-course of administration are as provided herein.

The administration may occur at least once, e.g., twice a week. In some embodiments, a composition described herein is administered twice a week. In some embodiments, a composition described herein is administered for 8 weeks. In some embodiments, a composition described herein is administered twice a week for 8 weeks. In some embodiments, a composition described herein is administered 16 times. In some embodiments, five compositions described herein are administered in a series twice a week for 2.5 weeks. In some embodiments, a composition described herein is administered for 4 weeks. In some embodiments, a composition described herein is administered twice a week for 4 weeks. In some embodiments, a composition described herein is administered 8 times. In some embodiments, a composition described herein is administered twice a week for 4.5 weeks. In some embodiments, a composition described herein is administered 9 times.

In some embodiments, the frequency of administration (and/or the dosage) may change, depending on the phase of treatment (e.g., a dose escalation phase or a tolerizing phase). In some embodiments, during a dose escalation phase, 0.9, 3, 9, 30, 60, 90, or 150 micrograms of the peptides (or an equivalent, such as a molar equivalent, thereof) described herein are administered at 5 time points. In some embodiments, 0.9 micrograms (or an equivalent, such as a molar equivalent, thereof) are administered at the first time point, 3 micrograms (or an equivalent, such as a molar equivalent, thereof) are administered at a second time point, 9 micrograms (or an equivalent, such as a molar equivalent, thereof) are administered at a third time point, 30 micrograms (or an equivalent, such as a molar equivalent, thereof) are administered at a fourth time point, and 90 micrograms (or an equivalent, such as a molar equivalent, thereof) are administered at a fifth time point.

In some embodiments, during a dose escalation phase, 3, 9, 30, 60, 90, or 150 micrograms of the peptides (or an equivalent, such as a molar equivalent, thereof) described herein are administered at 5 time points. In some embodiments, 3 micrograms (or an equivalent, such as a molar equivalent, thereof) are administered at the first time point, 9 micrograms (or an equivalent, such as a molar equivalent, thereof) are administered at a second time point, 30 micrograms (or an equivalent, such as a molar equivalent, thereof) are administered at a third time point, 60 micrograms (or an equivalent, such as a molar equivalent, thereof) are administered at a fourth time point, and 90 micrograms (or an equivalent, such as a molar equivalent, thereof) are administered at a fifth time point.

In some embodiments, during a dose escalation phase in a subject, such as one having a non-homozygous HLA-DQ2.5 genotype, 3, 9, 30, 60, 90, 150, 300, 450, 600, 750 or 900 micrograms of the peptides (or an equivalent, such as a molar equivalent, thereof) described herein are administered at 9 time points. In some embodiments, the non-homozygous HLA-DQ2.5 genotype is a heterozygous HLA-DQ2.5 genotype. In some embodiments, the heterozygous HLA-DQ2.5 genotype is HLA-DQ2.5/2.2, HLA-DQ2.5/7, or HLA-DQ2.5/8.

In some embodiments, during a tolerizing phase, 150 micrograms (or an equivalent, such as a molar equivalent, thereof) of the peptides described herein are administered. In some embodiments, 26.5 nmol of each of the first, second, and third peptides described herein are administered. In some embodiments, the administration is twice a week. In some embodiments, the administration is twice a week for 8 weeks.

In some embodiments, during a tolerizing phase, 300 micrograms (or an equivalent, such as a molar equivalent, thereof) of the peptides described herein are administered. In some embodiments, the administration is twice a week for 4 weeks. In some embodiments, during a tolerizing phase, a subject, such as one having a non-homozygous HLA-DQ2.5 genotype, is administered 450 micrograms, 600 micrograms, 750 micrograms, or 900 micrograms (or an equivalent, such as a molar equivalent, thereof) of the peptides described herein. In some embodiments, the administration is twice a week for 4 weeks. In some embodiments, the non-homozygous HLA-DQ2.5 genotype is a heterozygous HLA-DQ2.5 genotype. In some embodiments, the heterozygous HLA-DQ2.5 genotype is

HLA-DQ2.5/2.2, HLA-DQ2.5/7, or HLA-DQ2.5/8.

In some embodiments, any one of the treatment methods described herein comprises a any one of the tolerizing phases provided herein and any one of the dose escalation phases provided herein (preferably, prior to the tolerizing phase, in some embodiments).

### Kits

Another aspect of the disclosure relates to kits. In some embodiments, the kit comprises one or more compositions comprising the peptides as described herein. In some embodiments, the kit comprises at least two compositions at at least two different effective amounts described herein.

In some embodiments of any one of the kits described, the kit comprises one or more, such as all, of a first composition comprising 0.9 micrograms of one or more gluten peptides described herein, a second composition comprising 3 micrograms of one or more gluten peptides described herein, a third composition comprising 9 micrograms of one or more gluten peptides described herein, a fourth composition comprising 30 micrograms of one or more gluten peptides described herein, and a fifth composition comprising 90 micrograms of one or more gluten peptides described herein. In some embodiments of any one of the kits described, the kit further comprises a sixth composition comprising 150 micrograms of one or more gluten peptides described herein. In some embodiments of any one of the kits described, the one or more gluten peptides are a first peptide comprising the amino acid sequence

PFPQPELPY (SEQ ID NO: 4) and/or PQPELPYPQ (SEQ ID NO: 5); a second peptide comprising the amino acid sequence PFPQPEQPF (SEQ ID NO: 6) and/or PQPEQPFPW (SEQ ID NO: 7); and a third peptide comprising the amino acid sequence PIPEQPQPY (SEQ ID NO: 8) and/or EQPIPEQPQ (SEQ ID NO: 9). In some embodiments of any one of the kits described, one or more gluten peptides are a first peptide comprising the amino acid sequence ELQPFPQPELPYPQPQ (SEQ ID NO: 1), wherein the N-terminal glutamate is a pyroglutamate and the carboxyl group of the C-terminal glutamine is amidated; a second peptide comprising the amino acid sequence EQPFPQPEQPFPWQP (SEQ ID NO: 2), wherein the N-terminal glutamate is a pyroglutamate and the carboxyl group of the C-terminal proline is amidated; and a third peptide comprising the amino acid sequence

EPEQPIPEQPQPYPQQ (SEQ ID NO: 3), wherein the N-terminal glutamate is a pyroglutamate and the carboxyl group of the C-terminal glutamine is amidated.

In some embodiments of any one of the kits described, the kit comprises one or more, such as all, of a first composition comprising 0.3 micrograms of a first peptide and an equimolar amount of each of a second and a third peptide, a second composition comprising 1 microgram of a first peptide and an equimolar amount of each of a second and a third peptide, a third composition comprising 3 micrograms of a first peptide and an equimolar amount of each of a second and a third peptide, a fourth composition comprising 10 micrograms of a first peptide and an equimolar amount of each of a second and a third peptide, and a fifth composition comprising 30 micrograms of a first peptide and an equimolar amount of each of a second and a third peptide. In some embodiments of any one of the kits described, the kit further comprises a sixth composition comprising 50 micrograms of a first peptide and an equimolar amount of each of a second and a third peptide. In some embodiments, the first peptide comprises the amino acid sequence PFPQPELPY (SEQ ID NO: 4) and/or PQPELPYPQ (SEQ ID NO: 5); the second peptide comprises the amino acid sequence PFPQPEQPF (SEQ ID NO: 6) and/or PQPEQPFPW (SEQ ID NO: 7); and the third peptide comprises the amino acid sequence PIPEQPQPY (SEQ ID NO: 8) and/or EQPIPEQPQ (SEQ ID NO: 9). In some embodiments, the first peptide comprises the amino acid sequence ELQPFPQPELPYPQPQ (SEQ ID NO: 1), wherein the N-terminal glutamate is a pyroglutamate and the carboxyl group of the C-terminal glutamine is amidated; the second peptide comprises the amino acid sequence EQPFPQPEQPFPWQP (SEQ ID NO: 2), wherein the N-terminal glutamate is a pyroglutamate and the carboxyl group of the C-terminal proline is amidated; and the third peptide comprises the amino acid sequence EPEQPIPEQPQPYPQQ (SEQ ID NO: 3), wherein the N-terminal glutamate is a pyroglutamate and the carboxyl group of the C-terminal glutamine is amidated. In some embodiments of any one of the kits described, the kit comprises one or more, such as all, of a first composition comprising 1 microgram of a first peptide and an equimolar amount of each of a second and a third peptide, a second composition comprising 3 micrograms of a first peptide and an equimolar amount of each of a second and a third peptide, a third composition comprising 10 micrograms of a first peptide and an equimolar amount of each of a second and a third peptide, a fourth composition comprising 20 micrograms of a first peptide and an equimolar amount of each of a second and a third peptide, a fifth composition comprising 30 micrograms of a first peptide and an equimolar amount of each of a second and a third peptide, and a sixth composition comprising 50 micrograms of a first peptide and an equimolar amount of each of a second and a third peptide. In some embodiments of any one of the kits described, the kit further comprises a seventh composition comprising 100 micrograms of a first peptide and an equimolar amount of each of a second and a third peptide. In some embodiments, the first peptide comprises the amino acid sequence PFPQPELPY (SEQ ID NO: 4) and/or PQPELPYPQ (SEQ ID NO: 5); the second peptide comprises the amino acid sequence PFPQPEQPF (SEQ ID NO: 6) and/or PQPEQPFPW (SEQ ID NO: 7); and the third peptide comprises the amino acid sequence PIPEQPQPY (SEQ ID NO: 8) and/or EQPIPEQPQ (SEQ ID NO: 9). In some embodiments, the first peptide comprises the amino acid sequence ELQPFPQPELPYPQPQ (SEQ ID NO: 1), wherein the N-terminal glutamate is a pyroglutamate and the carboxyl group of the C-terminal glutamine is amidated; the second peptide comprises the amino acid sequence EQPFPQPEQPFPWQP (SEQ ID NO: 2), wherein the N-terminal glutamate is a pyroglutamate and the carboxyl group of the C-terminal proline is amidated; and the third peptide comprises the amino acid sequence EPEQPIPEQPQPYPQQ (SEQ ID NO: 3), wherein the N-terminal glutamate is a pyroglutamate and the carboxyl group of the C-terminal glutamine is amidated.

In some embodiments of any one of the kits described, the kit comprises compositions for any one of the tolerizing phases provided herein and any one of the dose escalation phases provided herein. The peptides can be contained within the same container or separate containers. In some embodiments of any one of the kits described, the peptide or peptides may be contained within the container(s) (e.g., dried onto the wall of the container(s)). In some embodiments of any one of the kits described, the peptides are contained within a solution separate from the container, such that the peptides may be added to the container at a subsequent time. In some embodiments of any one of the kits described, the peptides are in lyophilized form in a separate container, such that the peptides may be reconstituted and added to another container at a subsequent time. In some embodiments of any one of the kits described, the one or more compositions comprised within the kit are in a container that is suitable for intradermal injection (e.g., a device containing a needle such as a syringe). In some embodiments of any one of the kits described, the kit comprises a container that is suitable for intradermal injection (e.g., a device containing a needle such as a syringe).

In some embodiments of any one of the kits described, the kit further comprises instructions for reconstitution, mixing, administration, etc. In some embodiments of any one of the kits described, the instructions include the methods described herein. Instructions can be in any suitable form, e.g., as a printed insert or a label.

### Methods of Treatment

Aspects of the disclosure relate to use of the compositions described herein for treating a subject having, suspected of having or at risk of having Celiac disease.

As used herein, the terms “treat”, “treating”, and “treatment” include abrogating, inhibiting, slowing, or reversing the progression of a disease or condition, or ameliorating or preventing a clinical symptom of the disease (for example, Celiac disease). Treatment may include induction of immune tolerance (for example, to gluten or peptides thereof), modification of the cytokine secretion profile of the subject and/or induction of suppressor T cell subpopulations to secrete cytokines. Thus, a subject treated according to the disclosure preferably is able to eat at least wheat, rye, barley and, optionally, oats without a significant T cell response which would normally lead to symptoms of Celiac disease.

### Identifying Subjects for Treatment

In some embodiments, methods described herein comprise treating a subject who has Celiac disease. Thus, it may be desirable to identify subjects, such as subjects with Celiac disease, who are likely to benefit from administration of a composition described herein. It may also be desirable to monitor the treatment of the subjects with the compositions and methods provided herein. Any diagnostic method or other assay or combinations thereof are contemplated for identifying or monitoring such a subject. Any one of the methods provided herein can include identification and/or monitoring step(s). Exemplary methods include, but are not limited to, intestinal biopsy, serology (measuring the levels of one or more antibodies present in the serum), and genotyping (see, e.g., Husby S, Koletzko S, Korponay-Szabo I R, Mearin M L, Phillips A, Shamir R, Troncone R, Giersiepen K, Branski D, Catassi C et al: European Society for Pediatric Gastroenterology, Hepatology, and Nutrition guidelines for the diagnosis of coeliac disease. J Pediatr Gastroenterol Nutr 2012, 54(1):136-160. AND/OR Rubio-Tapia A, Hill I D, Kelly C P, Calderwood A H, Murray J A. ACG clinical guidelines: diagnosis and management of celiac disease. Am J Gastroenterol 2013; 108:656-76. AND/OR Ludvigsson J F, Leffler D A, Bai J C, Biagi F, Fasano A, Green P H, Hadjivassiliou M, Kaukinen K, Kelly C P, Leonard J N, Lundin K E, Murray J A, Sanders D S, Walker M M, Zingone F, Ciacci C. The Oslo definitions for coeliac disease and related terms. Gut 2012; 62:43-52.).

The presence of serum antibodies can be detected using methods known to those of skill in the art, e.g., by ELISA, histology, cytology, immunofluorescence or western blotting.

Such antibodies include, but are not limited to: IgA anti-endomysial antibody (IgA EMA), IgA anti-tissue transglutaminase 2 antibody (IgA tTG), IgA anti-deamidated gliadin peptide antibody (IgA DGP), and IgG anti-deamidated gliadin peptide antibody (IgG DGP). Deamidated gliadin peptide-IgA (DGP-IgA) and deamidated gliadin peptide-IgG (DGP-IgG) can be evaluated with commercial kits (e.g. INV 708760, 704525, and 704520, INOVA Diagnostics, San Diego, Calif.).

Subjects can be tested for the presence of the HLA-DQA and HLA-DQB susceptibility alleles encoding HLA-DQ2.5 (DQA1*05 and DQB1*02), DQ2.2 (DQA1*02 and DQB1*02) or DQ8 (DQA1*03 and DQB1*0302). Exemplary sequences that encode the DQA and DQB susceptibility alleles include HLA-DQA1*0501 (Genbank accession number: AF515813.1) HLA-DQA1*0505 (AH013295.2), HLA-DQB1*0201 (AY375842.1) or HLA-DQB1*0202 (AY375844.1). Methods of genetic testing are well known in the art (see, e.g., Bunce M, et al. Phototyping: comprehensive DNA typing for HLA-A, B, C, DRB1, DRB3, DRB4, DRB5 & DQB1 by PCR with 144 primer mixes utilizing sequence-specific primers (PCR-SSP). Tissue Antigens 46, 355-367 (1995); Olerup O, Aldener A, Fogdell A. HLA-DQB1 and DQA1 typing by PCR amplification with sequence-specific primers in 2 hours. Tissue antigens 41, 119-134 (1993); Mullighan C G, Bunce M, Welsh K I. High-resolution HLA-DQB1 typing using the polymerase chain reaction and sequence-specific primers. Tissue-Antigens. 50, 688-92 (1997); Koskinen L, Romanos J, Kaukinen K, Mustalahti K, Korponay-Szabo I, et al. (2009) Cost-effective HLA typing with tagging SNPs predicts celiac disease risk haplotypes in the Finnish, Hungarian, and Italian populations. Immunogenetics 61: 247-256.; and Monsuur A J, de Bakker P I, Zhernakova A, Pinto D, Verduijn W, et al. (2008) Effective detection of human leukocyte antigen risk alleles in celiac disease using tag single nucleotide polymorphisms. PLoS ONE 3:e2270). Subjects that have one or more copies of a susceptibility allele are considered to be positive for that allele. Detection of the presence of susceptibility alleles can be accomplished by any nucleic acid assay known in the art, e.g., by polymerase chain reaction (PCR) amplification of DNA extracted from the patient followed by hybridization with sequence-specific oligonucleotide probes or using leukocyte-derived DNA (Koskinen L, Romanos J, Kaukinen K, Mustalahti K, Korponay-Szabo I, Barisani D, Bardella M T, Ziberna F, Vatta S, Szeles G et al: Cost-effective HLA typing with tagging SNPs predicts Celiac disease risk haplotypes in the Finnish, Hungarian, and Italian populations. Immunogenetics 2009, 61(4):247-256; Monsuur A J, de Bakker P I, Zhernakova A, Pinto D, Verduijn W, Romanos J, Auricchio R, Lopez A, van Heel D A, Crusius J B et al: Effective detection of human leukocyte antigen risk alleles in Celiac disease using tag single nucleotide polymorphisms. PLoS ONE 2008, 3(5):e2270).

### EXAMPLES

### Example 1: Preparation of a 150 Microgram Dosage Composition of the First, Second, and Third Peptide

A dose of 150 μg the peptide composition was defined by there being 50 μg (26.5 nmol) of pure peptide 1, and an equimolar amount of peptide 2 and peptide 3. The molar equivalent of 50 μg peptide 1 was given by 50 μg/1889.3 g/mol=26.5 nmol. When preparing a solution containing 150 μg of the peptide composition, for the constituent peptides, the weight of each peptide was adjusted according to peptide purity and peptide content of the lyophilized stock material. For example, if the peptide 1 stock material had peptide purity of 98% and its peptide content was 90%, the weight of stock material yielding 50 μg peptide 1 was 50 μg/(peptide purity×peptide content)=50 ug/(0.98×0.90)=56.7 μg.

The molar amount of peptide 1 in 150 μg of the peptide composition was 26.5 nmol, and the weight of lyophilized peptide 2 stock material was therefore given by 26.5 nmol×1833.2 g/mol/(peptide purity x peptide content). For example, if peptide 2 peptide purity was 99%, and peptide content of 95%, the mass of stock required was 51.7 ug.

The molar amount of peptide 3 in 150 ug of the peptide composition was 26.5 nmol, and the weight of lyophilized peptide 3 stock material was therefore given by 26.5 nmol x 1886.2 g/mol /(peptide purity x peptide content). For example, if peptide 3 peptide purity was 98%, and peptide content of 92%, the mass of stock required was 55.4 ug.

0.9, 3, 9, 30, and 90 or any of the other microgram dosage compositions provided herein can be prepared similarly.

### Example 2: Dose Escalation Study

Objective: Determine tolerability of different escalating regimens followed by fixed dose and schedule for tolerance induction. Reduce adverse events and cytokine elevation associated with a large 1 time bolus (150 mcg) of peptide composition.

### Key Inclusion/Exclusion

patients having Celiac disease that are HLA-DQ2.5+

### Study Design

36 patients having Celiac disease that are HLA-DQ2.5+
Patients are administered doses of the peptide composition comprising peptide 1, 2, and 3 described herein (a first peptide comprising the amino acid sequence ELQPFPQPELPYPQPQ (SEQ ID NO: 1), wherein the N-terminal glutamate is a pyroglutamate and the carboxyl group of the C-terminal glutamine is amidated; a second peptide comprising the amino acid sequence EQPFPQPEQPFPWQP (SEQ ID NO: 2), wherein the N-terminal glutamate is a pyroglutamate and the carboxyl group of the C-terminal proline is amidated; and a third peptide comprising the amino acid sequence EPEQPIPEQPQPYPQQ (SEQ ID NO: 3), wherein the N-terminal glutamate is a pyroglutamate and the carboxyl group of the C-terminal glutamine is amidated) or placebo on the following dosage schedule:
Dose escalation regimen (or phase) for 5 doses at 0.9, 3, 9, 30, and 90 micrograms or placebo 2× a week for 2.5 weeks
Tolerizing regimen (or phase) of 150 micrograms twice a week for 8 weeks, follows dose escalation regimen

### Key Assessments

Primary Endpoint: Cytokine secretion
Secondary Endpoint: Symptoms

### Example 3. Further Dose Escalation Study Design

Primary Objective: To compare quantitative duodenal histology after a six week gluten challenge in HLA-DQ2.5+ patients with celiac disease on a gluten-free diet (GFD) who have been administered the peptide composition in Example 1 or placebo by intradermal injection.
Secondary Objective: To compare symptoms during a six week gluten challenge in HLA-DQ2.5+ patients with celiac disease on a gluten-free diet (GFD) who have been administered the peptide composition in Example 1 or placebo by intra-dermal injection.

### Study Design

The dose escalation regimen (or phase) and tolerizing regimen (or phase) described in Example 1 are carried out. A gluten escalation is performed over 14 days, followed by a 6 gram gluten challenge over 6 weeks. A biopsy is performed before the gluten escalation and after the 6 week challenge.

### Endpoints

Primary: VH:CrD—before vs after gluten challenge
Secondary: Clinical symptoms averaged for the last 2 weeks of subjects gluten challenge

### Example 4. Further Dose Escalation Study

### Primary Endpoint

Safety and tolerability

### Secondary Endpoint

Weekly GI symptoms per Gastrointestinal System Rating Scale
Assessment of plasma cytokine levels after sequential doses of gluten peptide composition

[symbol]

### Patients

Biopsy-confirmed, DQ2.5+ celiac disease patients on a GFD

### Dosing:

Titration Phase
   dose titration regimen to 300 micrograms for 2 weeks (3, 9, 30, 60, 90, 150, and 300 micrograms) or placebo
Tolerizing Phase
   dose at 300 micrograms twice per week or placebo for 4 weeks
Follow-up Phase
   4 weeks of follow up

### Example 5. Dose Escalation Study in Non-homozygotes for DQ2.5+

### Dosing:

Titration Phase
   dose titration regimen up to 900 micrograms for 4.5 weeks (3, 9, 30, 60, 90, 150, 300, 450, 600, 750, and up to 900 micrograms) or placebo
Maintenance Dosing Phase
   Dose at 300 micrograms (or up to 900 micrograms) or placebo twice per week for 4 weeks
Follow-up Phase
   4 weeks of follow up

### Example 6. Dose Escalation Study and Results

Primary Endpoint
Treatment emergent adverse events (TEAEs)

### Secondary Endpoints

Weekly Gastrointestinal Symptom Rating Scale (GSRS) scores, and relative change in plasma cytokine levels 4 hours after 150 microgram and higher doses. Plasma concentrations pre- and 45 min post- dose, and villous height to crypt depth ratio (VH:CrD) in 2nd part duodenal biopsies were assessed in Cohort 3.

[symbol]

### Patients

Biopsy-confirmed, DQ2.5+ celiac disease patients on a gluten-free diet

Patients were administered doses of peptide composition comprising peptide 1, 2, and 3 described herein (a first peptide comprising the amino acid sequence ELQPFPQPELPYPQPQ (SEQ ID NO: 1), wherein the N-terminal glutamate is a pyroglutamate and the carboxyl group of the C-terminal glutamine is amidated; a second peptide comprising the amino acid sequence EQPFPQPEQPFPWQP (SEQ ID NO: 2), wherein the N-terminal glutamate is a pyroglutamate and the carboxyl group of the C-terminal proline is amidated; and a third peptide comprising the amino acid sequence EPEQPIPEQPQPYPQQ (SEQ ID NO: 3), wherein the N-terminal glutamate is a pyroglutamate and the carboxyl group of the C-terminal glutamine is amidated) or placebo on the following dosage schedule.

### Dosing Regimen:

Cohorts 1 & 2 Titration Phase
   Twice-weekly dosing
   Initial up-dosing regimen of 30, 60, 90, 150, and 300 micrograms of peptide composition (or placebo)
   Amended to 3, 6, 9, 30, 60, 90, 150, and 300 micrograms of peptide composition (or placebo)
Cohort 3 Titration Phase
   Dose titration regimen up to 900 micrograms of peptide composition for 4.5 weeks (or placebo)
Maintenance Dosing Phase
   Cohorts 1 & 2: dose at 300 micrograms of peptide composition twice per week for 4 weeks (or placebo)
   Cohort 3: dose at maximum tolerated dose up to 900 micrograms of peptide composition (or placebo)
Follow-up Phase
   4 weeks of follow up

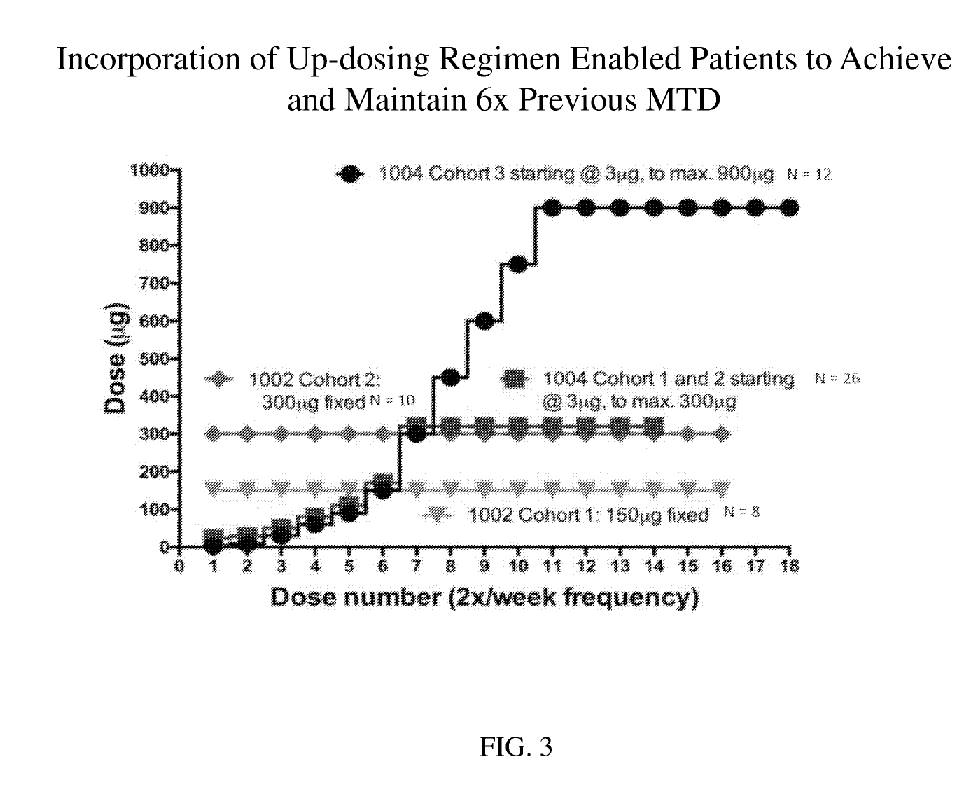

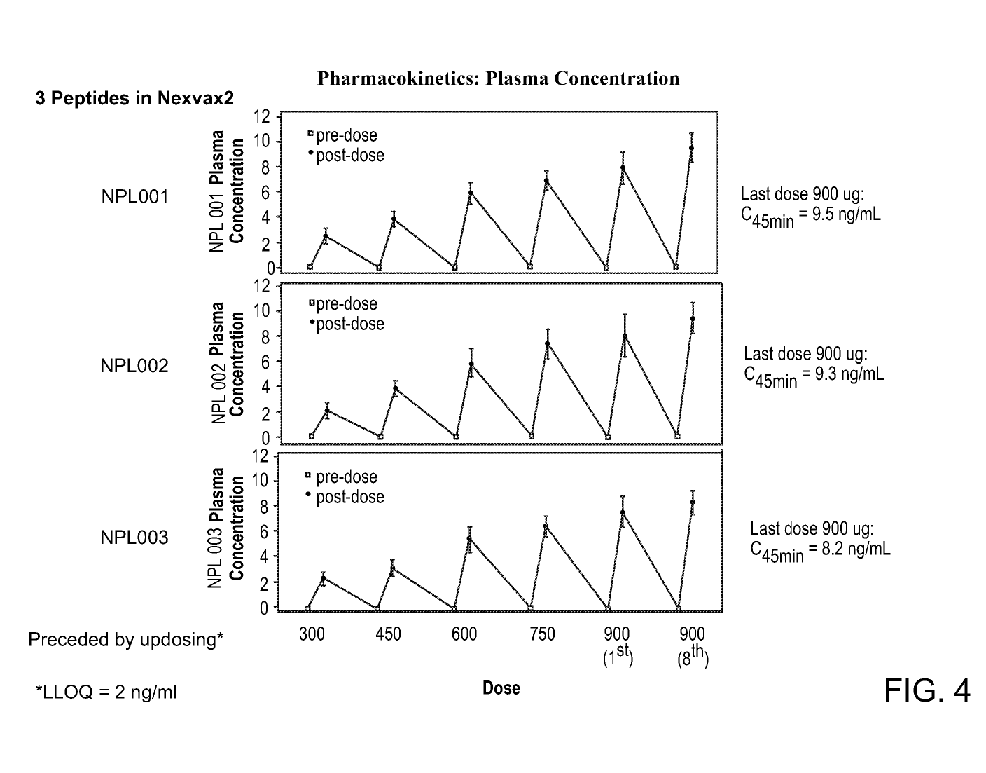

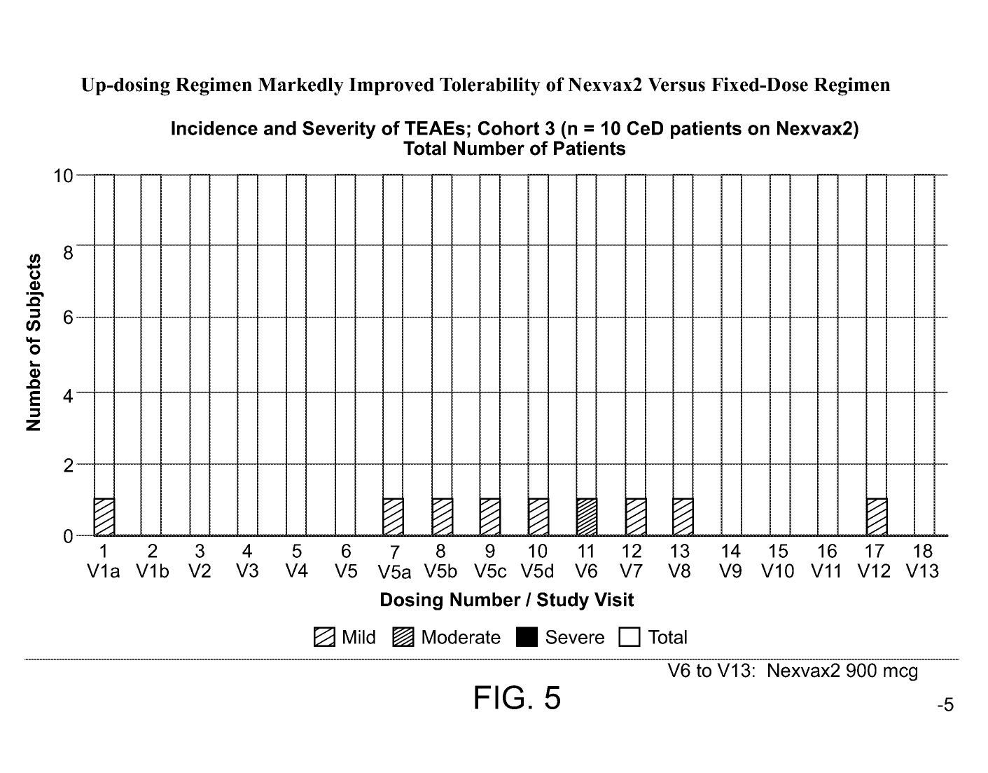

Thirty eight subjects (mean age 42 yr) were randomized 8:3, 10:5, or 10:2 to peptide composition or placebo in Cohorts 1, 2 and 3, respectively. All up-dosed patients tolerated and completed dosing at 900 micrograms. (FIG. 3). Both 300 microgram and 900 microgram doses were well-tolerated, including during the up-dosing titration. Treatment-related adverse events were mild and self-limiting. Pharmacokinetics of gluten peptides in plasma is shown in FIG. 4. The up-dosing regimen markedly improved the tolerability of peptide composition versus fixed-dose regimen (FIG. 5).

The second subject enrolled in Cohort 1 withdrew after 2 doses (30 micrograms and 60 micrograms) with severe abdominal pain, which led to a reduction in starting dose (3 micrograms). TEAEs with up-dosing from 3 micrograms and maintenance at 300 micrograms or 900 micrograms were mild or moderate apart from 1 subject in Cohort 2 who experienced a severe headache. Subjects who received placebo (n=9) had TEAEs similar to peptide composition treated subjects whose dosing started at 3 micrograms in Cohorts 1 (n=6) and 3 (n=10). Weekly mean GSRS decreased significantly each week after Week 3 of peptide composition treatment compared to baseline in Cohort 3 (p<0.05, Wilcoxon paired rank sum test).

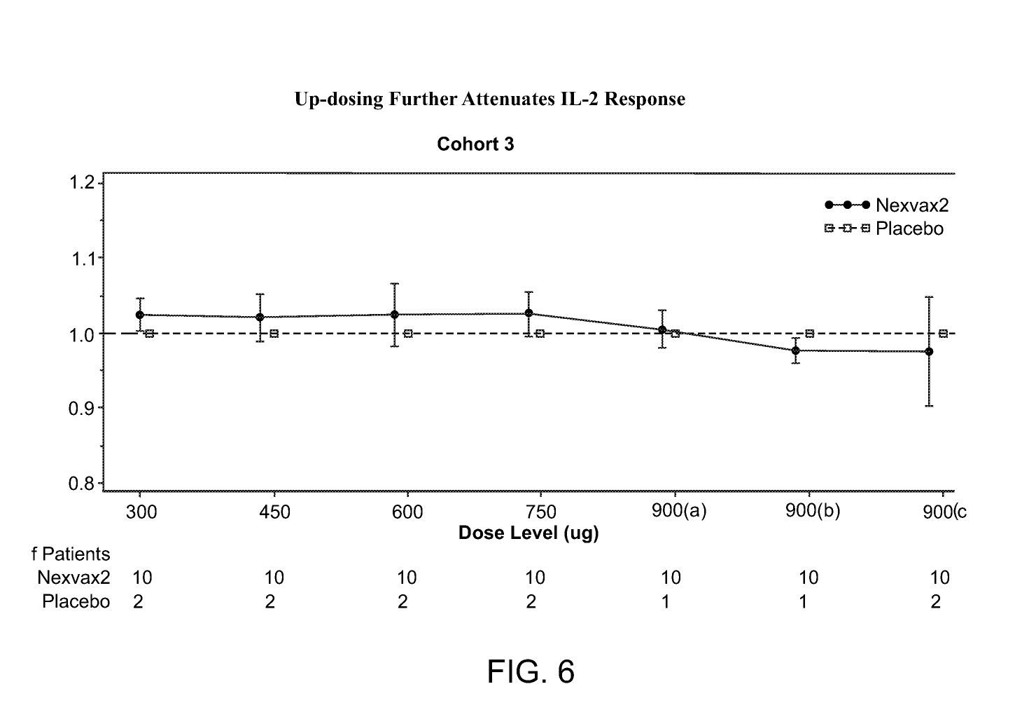

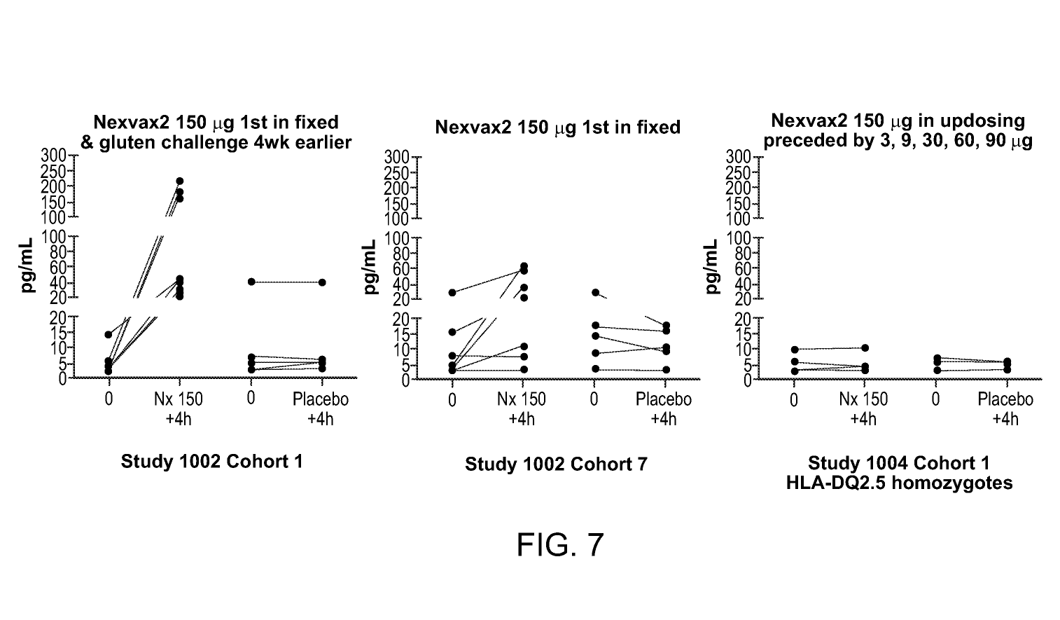

None of 38 cytokines were elevated in plasma at 4 h after ≥150 micrograms of peptide composition. No elevations in any cytokines or chemokines (e.g., IL-2, IL-8, MCP-1) at 4-hour post-dose following 150 microgram and subsequent dose levels were observed in any cohort. Up-dosing further attenuated the IL-2 response, as shown in FIG. 6. FIG. 7 is a series of graphs contrasting IL-2 release in plasma when comparing up-dosing (right panel) with fixed dosing (left and middle panel).

All peptide composition treated subjects in Cohort 3 had quantifiable, dose-dependent plasma levels of each of the peptides (˜9 ng/mL after 900 micrograms).

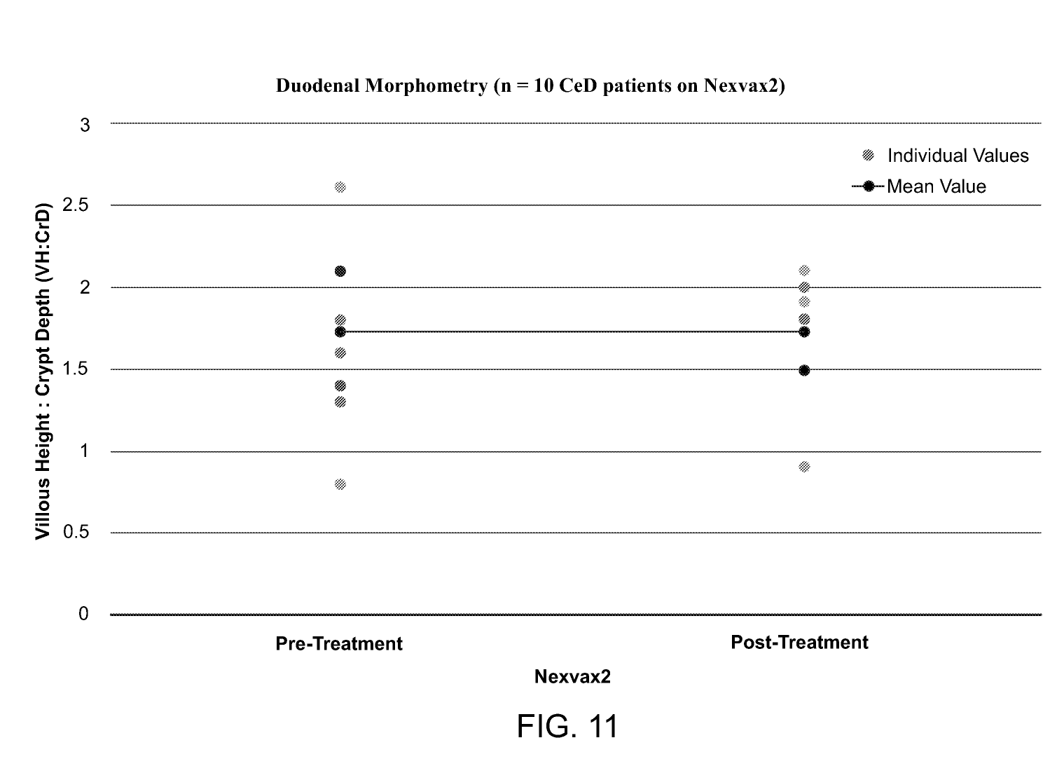

No overall change in duodenal histology compared to baseline was observed (FIG. 11). Mean (95% CI) duodenal VH:CrD was 1.7 (1.3-2.1) before and 1.7 (1.4-1.9) after treatment with peptide composition.

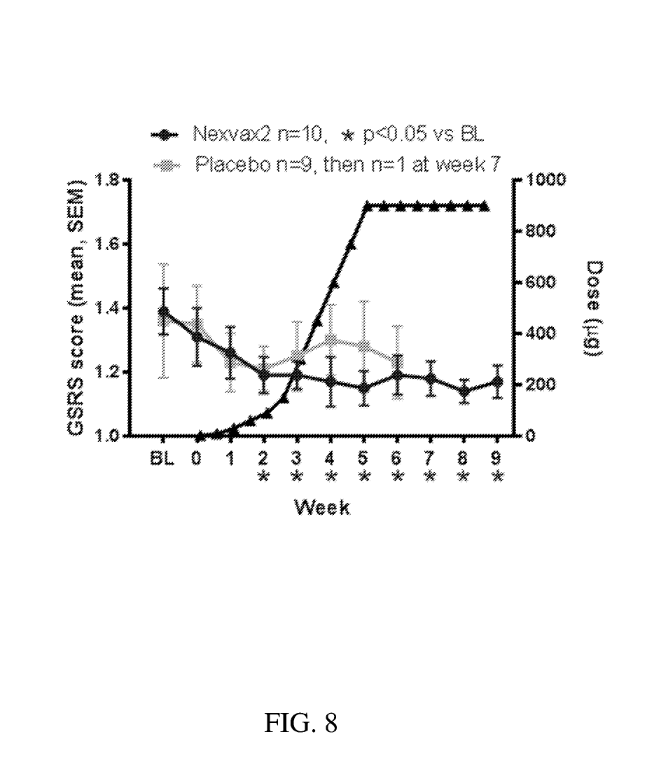

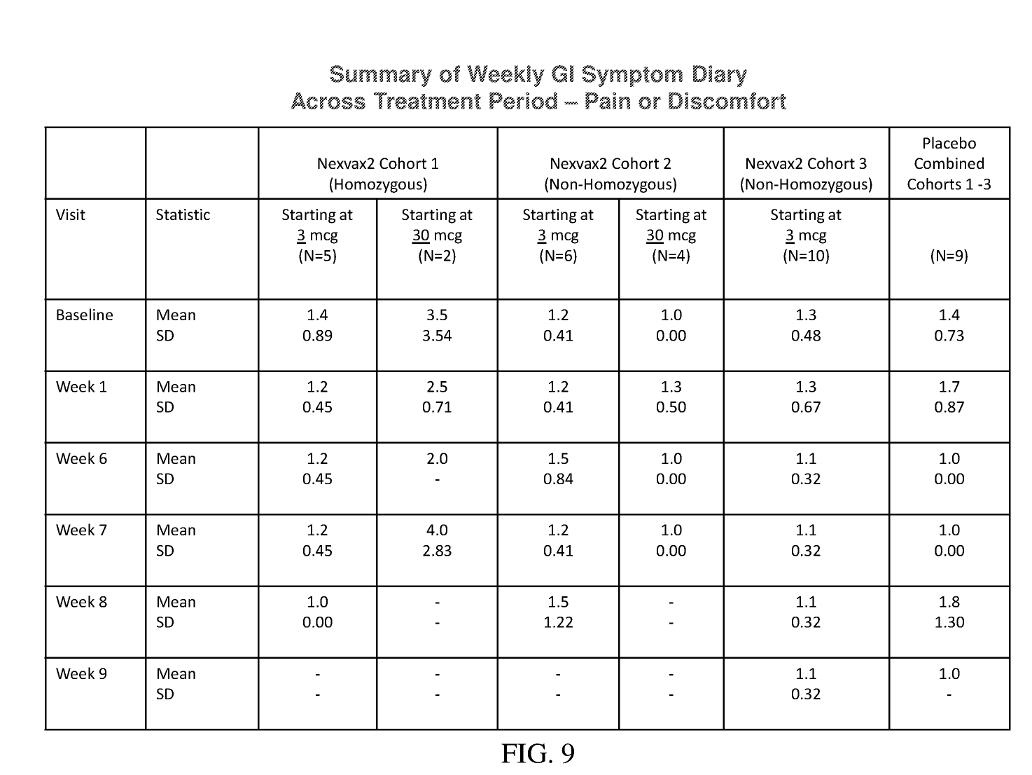

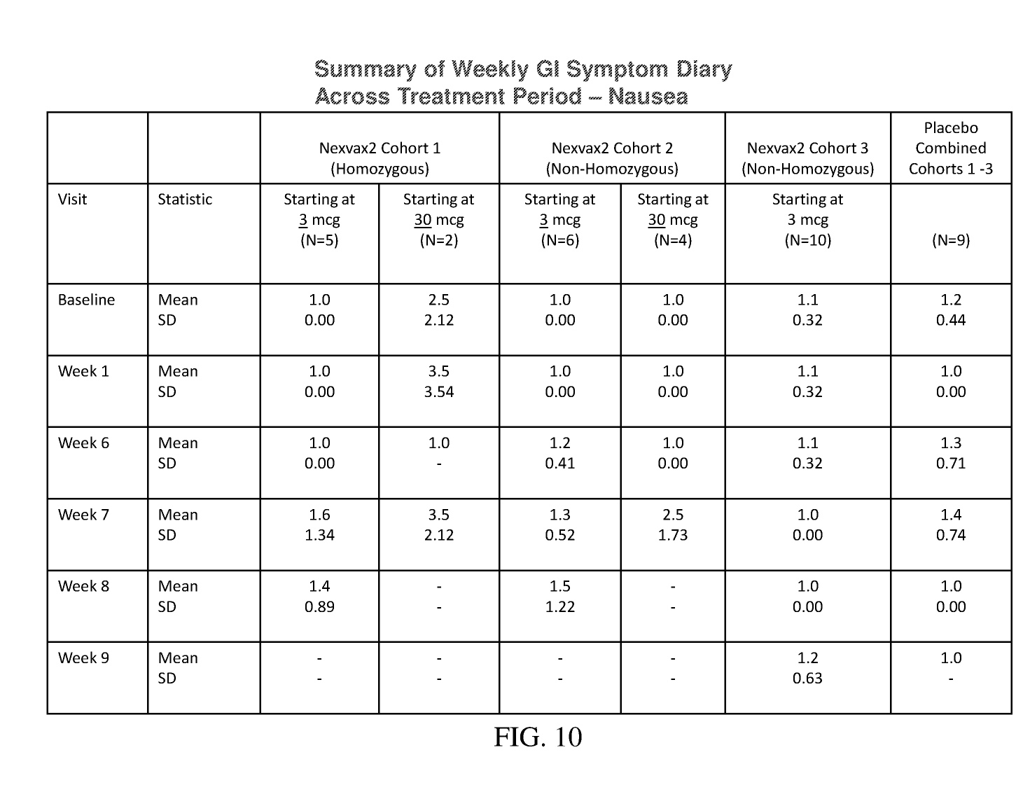

FIG. 8 shows that the treatment is associated with sustained reduction of symptoms per weekly GSRS (patient reported). FIG. 8 is a graph depicting Gastrointestinal Symptom Rating Scale (GSRS) score over time (lower numbers indicate lesser symptom severity). Overall symptom scores were measured at baseline and then weekly. There were 15 GI system domains. Placebo patients pooled all cohorts. Up-dosing began at 3 micrograms and the top dose was 900 micrograms. A significant reduction in symptoms compared to baseline was seen. No difference in symptoms between baseline and treatment period was seen in the placebo group. Tables summarizing the weekly GI symptom diary across treatment period related to pain or discomfort and the weekly GI symptom diary across treatment period related to nausea can be found respectively in FIGS. 9 and 10.

This example demonstrates that up-dosing enabled, among other things, achievement of a 900 microgram dose, which is 6 times higher versus a fixed-dose regimen. Up-dosing also enabled a well-tolerated regimen with a clean adverse events (AE) profile, which is significantly improved as compared to a fixed-dose regimen.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc. As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one,

A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A method for treating Celiac disease in a subject, the method comprising:
administering to the subject a first composition comprising 0.3, 1, 3, 10, 20, 30, or 50 micrograms of a first peptide and an equimolar amount of each of a second and a third peptide; and
subsequently administering to the subject a second composition comprising 50 or 100 micrograms of the first peptide and an equimolar amount of each of the second and third peptides,
wherein:
the first peptide comprises the amino acid sequence ELQPFPQPELPYPQPQ (SEQ ID NO: 1), wherein the N-terminal glutamate is a pyroglutamate and the C-terminal glutamine is amidated;
the second peptide comprises the amino acid sequence EQPFPQPEQPFPWQP (SEQ ID NO: 2), wherein the N-terminal glutamate is a pyroglutamate and the C-terminal proline is amidated; and
the third peptide comprises the amino acid sequence EPEQPIPEQPQPYPQQ (SEQ ID NO: 3), wherein the N-terminal glutamate is a pyroglutamate and the C-terminal glutamine is amidated.

2. The method of claim 1, wherein the first composition comprises 0.3 micrograms of the first peptide and an equimolar amount of each of the second and third peptides, and wherein the method further comprises:
administering to the subject a third composition comprising 1 micrograms of the first peptide and an equimolar amount of each of the second and third peptides; and
administering to the subject a fourth composition comprising 3 micrograms of the first peptide and an equimolar amount of each of the second and third peptides,
administering to the subject a fifth composition comprising 10 micrograms of the first peptide and an equimolar amount of each of the second and third peptides,
administering to the subject a sixth composition comprising 30 micrograms of the first peptide and an equimolar amount of each of the second and third peptides,
wherein the third, fourth, fifth and sixth composition are administered after administration of the first composition and before administration the second composition.

3. The method of claim 1 or 2, wherein the second composition comprises 50 micrograms of the first peptide and an equimolar amount of each of the second and third peptides.

4. The method of claim 1 or 2, wherein the second composition comprises 100 micrograms of the first peptide and an equimolar amount of each of the second and third peptides.

5. The method of any one of claims 2-4, wherein the first, third, fourth, fifth, and sixth composition are each administered once to the subject and the second composition is administered 16 times to the subject.

6. The method of claim 5, wherein the second composition is administered twice a week for 8 weeks.

7. The method of claim 1, wherein the first composition comprises 1 microgram of the first peptide and an equimolar amount of each of the second and third peptides, and wherein the method further comprises:
administering to the subject a third composition comprising 3 micrograms of the first peptide and an equimolar amount of each of the second and third peptides; and
administering to the subject a fourth composition comprising 10 micrograms of the first peptide and an equimolar amount of each of the second and third peptides,
administering to the subject a fifth composition comprising 20 micrograms of the first peptide and an equimolar amount of each of the second and third peptides,
administering to the subject a sixth composition comprising 30 micrograms of the first peptide and an equimolar amount of each of the second and third peptides,
wherein the third, fourth, fifth and sixth composition are administered after administration of the first composition and before administration the second composition.

8. The method of claim 7, wherein the first, third, fourth, fifth, and sixth composition are each administered once to the subject and the second composition is administered 8 times to the subject.

9. The method of claim 7, wherein the second composition is administered twice a week for 4 weeks.

10. The method of any one of the preceding claims, wherein the first, second and third peptides are in equimolar amounts in each of the first, second, third, fourth, fifth, and/or sixth composition(s).

11. The method of any of claims 1-10, wherein each of the first, second, third, fourth, fifth, and/or sixth composition(s) are/is administered intradermally.

12. The method of claim 11, wherein each of the first, second, third, fourth, fifth and/or sixth composition(s) are/is administered as a bolus by intradermal injection.

13. The method of any of claims 1-12, wherein each of the first, second, third, fourth, fifth and/or sixth composition(s) are/is formulated as a sterile, injectable solution.

14. The method of claim 13, wherein the sterile, injectable solution is sodium chloride.

15. The method of claim 14, wherein the sodium chloride is sterile sodium chloride 0.9% USP.

16. The method of any one of claims 1-15, wherein the subject is HLA-DQ2.5 positive.

17. The method of any one of claims 1-16, wherein the subject is on a gluten-free diet.

18. The method of any one of claims 1-17, wherein the method further comprises administering a composition comprising wheat, barley and/or rye to the subject after the second composition is administered.

19. A method for treating Celiac disease in a subject, such as one having a non-homozygous HLA-DQ2.5 genotype, the method comprising:
administering to the subject a first composition comprising 3, 9, 30, 60, 90, 150, 300, 450, 600, 750, or 900 micrograms of one or more gluten peptides; and subsequently administering to the subject a second composition comprising 300 micrograms or up to 900 micrograms of one or more gluten peptides.

20. The method of claim 19, wherein the method comprises administering to the subject a first composition comprising 30 micrograms of one or more gluten peptides, a third composition comprising 60 micrograms of one or more gluten peptides, a fourth composition comprising 90 micrograms of one or more gluten peptides, a fifth composition comprising 150 micrograms of one or more gluten peptides, a sixth composition comprising 300 micrograms of one or more gluten peptides, a seventh composition comprising 450 micrograms of one or more gluten peptides, an eighth composition comprising 600 micrograms of one or more gluten peptides, a ninth composition comprising 750 micrograms of one or more gluten peptides and subsequently administering to the subject a tenth composition comprising 900 micrograms of one or more gluten peptides, wherein the third, fourth, fifth, sixth, seventh, eighth, ninth, and tenth compositions are administered after administration of the first composition and before administration the second composition.

21. The method of claim 19 or 20, wherein the second composition comprises 300 micrograms of one or more gluten peptides.

22. The method of claim 19 or 20, wherein the second composition comprises up to 900 micrograms of one or more gluten peptides.

23. The method of any one of claims 19-22, wherein the non-homozygous HLA-DQ2.5 genotype is a heterozygous HLA-DQ2.5 genotype.

24. The method of any one of claims 19-22, wherein the heterozygous HLA-DQ2.5 genotype is HLA-DQ2.5/2.2, HLA-DQ2.5/7, or HLA-DQ2.5/8.

25. The method of any one of claims 19-24, wherein first, third, fourth, fifth sixth, seventh, eighth, ninth, and tenth compositions are administered over 4.5 weeks, with administrations occurring at a frequency of twice a week.

26. The method of any one of claims 19-24, wherein the second composition is administered twice a week for 4 weeks.

27. A method for treating Celiac disease in a subject, the method comprising administering one or more gluten peptide compositions according to any one of the dosing regimens provided herein, such as in the Examples or Figures.

28. The method of claim 27, wherein the one or more gluten peptide compositions comprises any one of the gluten peptide compositions provided herein.

29. The method of claim 28, wherein the one or more gluten peptide compositions comprises peptides 1, 2 and 3 of Example 6.

30. The method of any one of claims 27-29, wherein the subject is any one of the subjects provided herein.

31. The method of claim 30, wherein the subject has a non-homozygous HLA-DQ2.5 genotype.

32. A method for treating Celiac disease in a subject, the method comprising administering one or more gluten peptide compositions according to any one of the titration or dose escalation regimens or phases as provided herein and any one of the tolerizing or maintenance regimens or phases as provided herein, such as in any one of the Examples or Figures.

33. One or more gluten peptide compositions for performing a method as in any one of claims 1-32.

34. A kit comprising one or more gluten peptide compositions for performing a method as in any one of claims 1-32.


SEQUENCE LISTING (9 sequences)

SEQ ID NO: 1 - protein, 16 aa, Artificial Sequence
  Synthetic Polypeptide
ELQPFPQPELPYPQPQ

SEQ ID NO: 2 - protein, 15 aa, Artificial Sequence
  Synthetic Polypeptide
EQPFPQPEQPFPWQP

SEQ ID NO: 3 - protein, 16 aa, Artificial Sequence
  Synthetic Polypeptide
EPEQPIPEQPQPYPQQ

SEQ ID NO: 4 - protein, 9 aa, Artificial Sequence
  Synthetic Polypeptide
PFPQPELPY

SEQ ID NO: 5 - protein, 9 aa, Artificial Sequence
  Synthetic Polypeptide
PQPELPYPQ

SEQ ID NO: 6 - protein, 9 aa, Artificial Sequence
  Synthetic Polypeptide
PFPQPEQPF

SEQ ID NO: 7 - protein, 9 aa, Artificial Sequence
  Synthetic Polypeptide
PQPEQPFPW

SEQ ID NO: 8 - protein, 9 aa, Artificial Sequence
  Synthetic Polypeptide
PIPEQPQPY

SEQ ID NO: 9 - protein, 9 aa, Artificial Sequence
  Synthetic Polypeptide
EQPIPEQPQ